SEQUENCE LISTING (21 sequences)

SEQ ID NO: 1 - protein, 805 aa, Homo sapiens [sequence omitted]

SEQ ID NO: 2 - protein, 715 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLG

SEQ ID NO: 3 - protein, 723 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 4 - protein, 348 aa, Homo sapiens
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 5 - protein, 8 aa, synthetic construct
  REGION 1..8: note = linker sequence
GGGSGGGS

SEQ ID NO: 6 - protein, 18 aa, Homo sapiens
MKWVTFISLLFLFSSAYS

SEQ ID NO: 7 - protein, 130 aa, Homo sapiens
VALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 8 - protein, 1097 aa, synthetic construct
  REGION 1..1097: note = ACE2-IgM fusion protein
MKWVTFISLLFLFSSAYSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGSGGGSIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 9 - protein, 879 aa, synthetic construct
  REGION 1..879: note = ACE2 cm4 fusion protein
MKWVTFISLLFLFSSAYSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGSGGGSVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 10 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 11 - protein, 977 aa, synthetic construct
  REGION 1..977: note = ACE2-IgG2 fusion protein
MKWVTFISLLFLFSSAYSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGSGGGSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 12 - protein, 588 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVG

SEQ ID NO: 13 - protein, 1273 aa, Severe acute respiratory syndrome-related coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - protein, 13 aa, Homo sapiens
ESKYGPPCPPCPA

SEQ ID NO: 15 - protein, 11 aa, Homo sapiens
DKTHTCPPCPA

SEQ ID NO: 16 - protein, 18 aa, Homo sapiens
ERKCCVECPPCPAPPVAG

SEQ ID NO: 17 - protein, 348 aa, Homo sapiens; identical to SEQ ID NO: 4

SEQ ID NO: 18 - protein, 330 aa, Homo sapiens
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGK

SEQ ID NO: 19 - protein, 236 aa, Homo sapiens
QDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 20 - protein, 218 aa, Homo sapiens
QDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGK

SEQ ID NO: 21 - protein, 112 aa, Homo sapiens
VALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGK

(19) United States
(12) Patent Application Publication
(10) Pub. No.: US 2025/0082737 A1
(43) Pub. Date: Mar. 13, 2025

(54) ACE2 FUSION PROTEINS AND USES THEREOF

(71) Applicant: Formycon AG, Martinsried/Planegg (DE)

(72) Inventors: Johann Sebastian BUCHNER, Ihrlerstein (DE); Hristo Lyubomirov SVILENOV, Eke (BE)

(73) Assignee: Formycon AG, Martinsried/Planegg (DE)

(21) Appl. No.: 18/292,773

(22) PCT Filed: Jul. 29, 2022
(86) PCT No.: PCT/EP2022/071308

§ 371 Date: Jan. 26, 2024

(30) Foreign Application Priority Data
Jul. 30, 2021 (EP) 21188832.6
Jun. 29, 2022 (EP) 22182062.4

(51) Int. Cl. A61K 38/48; A61P 31/14; C12N 9/48

(52) U.S. Cl. CPC A61K 38/4813; A61P 31/14; C12N 9/485; C12Y 304/17023; C07K 2319/30

(57) ABSTRACT

The present invention relates to fusion proteins of ACE2 with IgM or IgG2 Fc and the medical use of these fusion proteins, in particular in the prevention or treatment of infections with coronaviruses such as SARS-CoV-2.

### FIELD OF THE INVENTION

The present invention relates to fusion proteins of ACE2 with the Fc part of IgM or IgG2 and the medical use of these fusion proteins, in particular in the prevention or treatment of infections with coronaviruses such as SARS-CoV-2.

### BACKGROUND OF THE INVENTION

ACE2 (angiotensin converting enzyme 2) is a key metalloprotease of the renin-angiotensin system with a catalytic zinc atom in the centre (Donoghue et al. (2002) Circ. Res. 87: e1-e9). Full-length ACE2 consists of an N-terminal extracellular peptidase domain, a collectrin-like domain, a single transmembrane helix and a short intracellular segment. It acts to cleave Angiotensin II to produce Angiotensin (1-7) and Angiotensin I to produce Angiotensin (1-9) which is then processed by other enzymes to become Angiotensin (1-7). ACE2 acts to lower the blood pressure and counters the activity of ACE in order to maintain a balance in the Ras/MAS system. Accordingly, it is a promising target for the treatment of cardiovascular diseases.

Recently, ACE2 has gained much attention as a receptor for coronaviruses and in particular the novel coronavirus SARS-CoV-2. SARS-CoV-2 is a coronavirus which was first discovered in December 2019 in Wuhan, China, but spread rapidly all over the world, leading to a world-wide pandemic. On 15 Jul. 2021, the Johns Hopkins University counted more than 188 millions of confirmed infections worldwide, causing the death of more than 4 million people. The pandemic led to several lock-downs in many countries with very significant economic and social effects.

It was shown that ACE2 functions as a receptor for SARS-CoV (Li et al. (2003) Nature 426: 450-454; Prakabaran et al. (2004) Biochem. Biophys. Res. Comm. 314: 235-241) and for SARS-CoV-2 (Yan et al. (2020) Science 367: 1444-1485). Further, entry of SARS-CoV-2 into the respiratory cells depends on ACE2 and the serine protease TMPRSS2 (Hoffmann et al. (2020) Cell 181: 1-10).

In view of the important role of ACE2 for virus entry into the cell, it was proposed to use soluble ACE2 for blocking SARS binding to the cell (WO 2005/032487; WO 2006/122819). The same approach was also suggested for the treatment of infections with SARS-CoV-2 (Kruse (2020) F1000Res. 9:72). Clinical trials with a soluble form of ACE2 in the treatment of infections with SARS-CoV-2 have been initiated by the company Apeiron (Pharmazeutische Zeitung, 10 Apr. 2020) and the first results showed that one patient with severe Covid-19 caused by SARS-CoV-2 recovered fast upon treatment with soluble ACE2 (Zoufaly et al. (2020) The Lancet Respiratory Medicine 8: 1154-1158).

However, isolated receptor domains are typically characterized by a low stability and plasma half-life. For the soluble form of ACE2 a dose-dependent terminal half-life of 10 hours was shown (Haschke et al. (2013) Clin. Pharmacokinet. 52: 783-792). In view of these results, it was decided to administer the soluble form of ACE2 as twice-daily infusion in a later study (Khan et al. (2017) Critical Care 21: 234). However, an administration of more than one time per day is inconvenient both for the patient and the medical personnel.

A fusion protein of ACE2 consisting of the extracellular domain of either enzymatically active or enzymatically inactive ACE2 linked to the Fc domain of human IgG1 was constructed and tested. It was shown that both constructs potently neutralized both SARS-CoV and SARS-CoV-2 and inhibited S (Spike) protein-mediated fusion (Lei et al. (2020) Nature Communications 11: 2070). Further, a fusion protein called COVIDTRAP™ or STI-4398 was developed for clinical testing by the company Sorrento Therapeutics (see https://www.globenewswire.com/news-release/2020/03/20/2003957/0/en/SORRENTO-DEVELOPS-STI-4398-COVIDTRAP-PROTEIN-FOR-POTENTIAL-PREVENTION-AND-TREATMENT-OF-SARS-COV-2-CORONAVIRUS-DISEASE-COVID-19.html). An ACE2 fusion protein with the Fc domain of IgG1 called HLX71 is also tested by the company Shanghai Henlius Biotech (see Henlius ACE2-Fc Fusion Protein HLX71 Received IND Approval from US FDA-Media). Liu et al. (2020) Int. J. Biol. Macromol. 165: 1626-1633 describe fusion proteins of wild-type ACE2 and nine ACE2 mutants affecting catalytic activity of ACE2 with the Fc region of human IgG1. Similarly, Ferrari et al. (2021), available at Characterisation of a novel ACE2-based therapeutic with enhanced rather than reduced activity against SARS-CoV2 variants| bioRxiv, describe a fusion protein of enzymatically inactive ACE2 with the Fc domain of human IgG1. Castilho et al. (2021) Biotechnology Journal 16: 2000566 describe the production of ACE2-Fc in glycoengineered Nicotiana benthamiana. However, the interaction of the Fc domain of human IgG1 with Fc gamma receptors on immune cells may enhance the virus infection (Perlman and Dandekar (2005) Nat. Rev. Immunol. 5(12): 917-927; Chen et al. (2020) Current Tropical Medicine Reports 3:1-4).

Tada et al. (2020), available at https://www.biorxiv.org/content/10.1101/2020.09.16.300319v1.full, disclose an “ACE2 microbody” in which the extracellular domain of catalytically inactive ACE2 is fused to the CH3 domain of an Fc part of the immunoglobulin heavy chain.

Iwanaga et al. (2020), available at Novel ACE2-IgG1 fusions with improved in vitro and in vivo activity against SARS-CoV2| bioRxiv discloses ACE2-human IgG1 fusion proteins with mutations in the catalytic domain of ACE2 and a LALA mutation in the Fc part which abrogates Fcγ receptor binding, but retains FcRn binding.

Svilenov et al. (2020), available at Efficient inhibition of SARS-CoV-2 strains by a novel ACE2-IgG4-Fc fusion protein with a stabilized hinge region|bioRxiv and WO 2021/234160 describe ACE2 fusion proteins with the Fc domain of IgG4. Further ACE2 fusion protein constructs are described in WO 2022/090469.

Hence, there is still a need for agents which can be used for the treatment and/or prevention of infections with coronaviruses, in particular with SARS-CoV-2.

### SUMMARY OF THE INVENTION

The present invention provides a fusion protein comprising a first part comprising a fragment of human ACE2 or a variant of said fragment, said human ACE2 having the amino acid sequence according to SEQ ID NO: 1, and a second part comprising the Fc portion of human IgM or a fragment or variant of the Fc portion of human IgM.

The Fc portion of human IgM may have the amino acid sequence according to SEQ ID NO: 4.

The fragment of human ACE2 may consist of the amino acid sequence according to SEQ ID NO: 2 or may be the extracellular domain of ACE2 consisting of the amino acid sequence according to SEQ ID NO: 3.

The first part and the second part of the fusion protein may be linked by a linker sequence such as a linker sequence consisting of glycine and serine residues. In one embodiment, the linker sequence has the amino acid sequence according to SEQ ID NO: 5.

In one embodiment, the fusion protein further comprises a signal peptide, which may comprise the amino acid sequence according to SEQ ID NO: 6.

In one embodiment, the fragment of the Fc portion of human IgM consists of the amino acid sequence according to SEQ ID NO: 7.

In one embodiment, the fusion protein has the amino acid sequence according to SEQ ID NO: 8 or 9.

In one embodiment, the present invention provides a fusion protein comprising a first part comprising a fragment of human ACE2 or a variant of said fragment, said human ACE2 having the amino acid sequence according to SEQ ID NO: 1, and a second part comprising the Fc portion of human IgG2 or a variant of the Fc portion of human IgG2, wherein the first part and the second part are linked by a linker sequence.

In one embodiment, the Fc portion of human IgG2 comprises the amino acid sequence according to SEQ ID NO: 10.

The fragment of human ACE2 may consist of the amino acid sequence according to SEQ ID NO: 2 or may be the extracellular domain of ACE2 consisting of the amino acid sequence according to SEQ ID NO: 3.

The first part and the second part of the fusion protein may be linked by a linker sequence such as a linker sequence consisting of glycine and serine residues. In one embodiment, the linker sequence has the amino acid sequence according to SEQ ID NO: 5.

In one embodiment, the fusion protein has the amino acid sequence according to SEQ ID NO: 11.

In one embodiment, the variant of the human ACE2 fragment is an enzymatically inactive variant of human ACE2 which may comprise a H374N and a H378N mutation, the numbering referring to SEQ ID NO: 1.

The variant of the human ACE2 fragment may comprise an amino acid substitution at leucine 584, the numbering referring to SEQ ID NO: 1 and/or at least one amino acid substitution at at least one residue selected from lysine 619, arginine 621, lysine 625, arginine 697, lysine 702, arginine 705, arginine 708, arginine 710 and arginine 716, the numbering referring to SEQ ID NO: 1.

In one embodiment the variant of the human ACE2 fragment comprises amino acid substitutions at lysine 619, arginine 621, lysine 625, arginine 697, lysine 702, arginine 705, arginine 708, arginine 710 and arginine 716, the numbering referring to SEQ ID NO: 1.

In one embodiment the variant of the human ACE2 fragment comprises a S645C mutation, the numbering referring to SEQ ID NO: 1.

The present invention also relates to a nucleic acid molecule comprising a nucleic acid sequence encoding said fusion protein, an expression vector comprising said nucleic acid molecule and a host cell comprising said nucleic acid molecule or said expression vector.

Further, the present invention relates to a method for producing said fusion protein, comprising culturing said host cell in a suitable culture medium.

The present invention also relates to a multimer of the ACE2 fusion protein with the Fc portion of human IgM or a fragment or variant of the Fc portion of human IgM.

The present invention also relates to a hexamer of the ACE2 fusion protein with the Fc portion of human IgM or a fragment or variant of the Fc portion of human IgM.

The present invention also relates to said fusion protein, said multimer or said hexamer for medical use, in particular for use in preventing and/or treating an infection with a coronavirus binding to ACE2, or for use in diagnosis.

In one embodiment, the coronavirus binding to ACE2 is selected from the group consisting of SARS, SARS-CoV-2 and NL-63, preferably is SARS-CoV-2.

Said fusion protein or said multimer may be administered in combination with an anti-viral agent which may be selected from the group consisting of remdesivir, arbidol HCl, ritonavir, lopinavir, darunavir, ribavirin, chloroquin and derivatives thereof, nitazoxanide, camostat mesilate, tocilizumab, siltuximab, sarilumab and baricitinib phosphate.

The present invention also relates to said fusion protein or said multimer for use in treating hypertension (including high blood pressure), congestive heart failure, chronic heart failure, acute heart failure, contractile heart failure, myocardial infarction, arteriosclerosis, kidney failure, renal failure, Acute Respiratory Distress Syndrome (ARDS), Acute Lung Injury (ALI), chronic obstructive pulmonary disease (COPD), pulmonary hypertension, renal fibrosis, chronic renal failure, acute renal failure, acute kidney injury, inflammatory bowel disease and multi-organ dysfunction syndrome.

The present invention also relates to a pharmaceutical composition comprising said fusion protein or said multimer and a pharmaceutically acceptable carrier or excipient. The pharmaceutical composition may further comprise an anti-viral agent.

### BRIEF DESCRIPTION OF THE DRAWINGS

FIG. 1: Analysis of quaternary structure and oligomeric state of the ACE2 fusion protein with the Fc domain of IgM (a), the ACE2 fusion protein with a truncated Fc domain of IgM (cm4) according to SEQ ID NO: 7 (b) and the ACE2 fusion protein with the Fc domain of IgG2 (c)

FIG. 2: Binding to the receptor-binding domain of SARS-CoV2 of the hexameric ACE2 fusion protein with the Fc domain of IgM (a), the ACE2 fusion protein with a truncated Fc domain of IgM (cm4) according to SEQ ID NO: 7 (b), the ACE2 fusion protein with the Fc domain of IgG2 (c) and the homodimeric ACE2 fusion protein with the Fc domain of IgM (d).

FIG. 3: Oligomeric state of ACE2-IgM fusions
a) SEC-MALS chromatogram with calculated molecular masses of the hexameric ACE2 fusion protein with the Fc domain of IgM (left peak) and the corresponding monomers (right peak)
b) SEC-MALS chromatogram with calculated rms radius of the hexameric ACE2 fusion protein with the Fc domain of IgM

FIG. 4: Neutralization efficiency of ACE2-IgM-Fc hexamer in a commercial pseudovirus assay with the Omicron variant. The IC50 from the fit is 6.6 ng/mL (95% confidence interval 5.7 to 7.6 ng/mL). The values are mean of triplicates with standard deviations.

FIG. 5: Cumulative severity score from lungs of SARS-CoV-2-infected golden Syrian hamsters

Cumulative severity score from H&E-stained slides of lungs from SARS-CoV-2-infected golden Syrian hamsters following prophylactic treatment via the intranasal route with PBS control, Casirivimab, or ACE2-IgM at 4 dpi. Statistical analysis was performed using non-parametric Mann Whitney U-test, *P<0.05. **P<0.01. The graph represents mean±SD. The dotted line represents the mean lung score of 1.25 in healthy, untreated, non-infected animals.

### DETAILED DESCRIPTION OF THE INVENTION

The present invention as illustratively described in the following may suitably be practiced in the absence of any element or elements, limitation or limitations, not specifically disclosed herein.

The present invention will be described with respect to particular embodiments, but the invention is not limited thereto, but only by the claims.

Where the term “comprising” is used in the present description and claims, it does not exclude other elements. For the purposes of the present invention, the term “consisting of” is considered to be a preferred embodiment of the term “comprising”. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. a cell or organism is defined to be obtainable by a specific method, this is also to be understood to disclose a cell or organism which is obtained by this method.

Where an indefinite or definite article is used when referring to a singular noun, e.g. “a”, “an” or “the”, this includes a plural of that noun unless something else is specifically stated.

As discussed above, the present invention provides a fusion protein comprising a first part comprising a fragment of human ACE2 or a variant of said fragment and a second part comprising the Fc portion of human IgM or a variant or fragment of the Fc portion of human IgM or the Fc portion of human IgG2 or a variant or fragment of the Fc portion of human IgG2. In view of the literature (e.g. Tada et al. (2020), available at https://www.biorxiv.org/content/10.1101/2020.09.16.300319v1.full) it is assumed that this fusion protein has a higher affinity to the Spike protein of SARS-CoV-2 than a soluble ACE2 dimer without the Fc portion. It Is also assumed that the disulfide bridge between the Fc portions stabilizes the binding of the fusion proteins to their target, such as the Spike protein of SARS-CoV-2. As shown herein, a hexamer of homodimer of the ACE2 fusion protein with the Fc portion of human IgM shows a stronger binding to the SARS-CoV2 receptor-binding domain than the homodimer and is able to efficiently neutralize the SARS-CoV2 Omicron variant.

A “fusion protein” is a protein which is formed by at least two polypeptide parts which are not naturally linked with each other. The two polypeptide parts are linked by a peptide bond and optionally a linker molecule is inserted between the two polypeptide parts. The two polypeptide parts are transcribed and translated as a single molecule. The fusion protein typically has functionalities derived from both polypeptide parts. In the context of the present invention, the fusion protein retains the binding properties of ACE2, in particular the binding of viruses such as coronaviruses, and the increased half-life and Fc receptor binding conferred by the Fc portion of human IgM or IgG2.

The term “human ACE2” refers to angiotensin converting enzyme 2 derived from a human subject. The full-length sequence of human ACE2 has 805 amino acids. It comprises a signal peptide, an N-terminal extracellular peptidase domain followed by a collectrin-like domain, a single transmembrane helix and a short intracellular segment. The full-length sequence of human ACE2 is depicted in SEQ ID NO:1. Unless indicated otherwise, the amino acid numbering used herein refers to the numbering of the full-length sequence of human ACE2 according to SEQ ID NO: 1. The extracellular domain of human ACE2 consists of amino acids 18 to 740 of SEQ ID NO: 1 and is shown in SEQ ID NO: 3.

The term “fragment of human ACE2” refers to a polypeptide which lacks one or more amino acids compared to the full-length sequence of human ACE2 according to SEQ ID NO:1. The fragment of human ACE2 is capable of binding to the S protein of at least one coronavirus, in particular to the S protein of SARS-CoV-2. The binding of a fragment of human ACE2 to the S protein of at least one coronavirus, in particular to the S protein of SARS-CoV-2, can be determined in an ELISA assay in which the S protein is immobilized on a substrate and contacted with the fragment of human ACE2 and the interaction between the S protein and the fragment of human ACE2 is detected. Alternatively, the binding of a fragment of human ACE2 to the S protein of at least one coronavirus, in particular to the S protein of SARS-CoV-2, can be determined by surface plasmon resonance, e.g. as described in Shang et al. (2020) Nature doi: 10.1038/s41586-020-2179-y; Wrapp et al. (2020) Science 367(6483): 1260-1263; Lei et al. (2020) Nature Communications 11(1): 2070. In a further alternative, the binding of a fragment of human ACE2 to the S protein of at least one coronavirus, in particular to the S protein of SARS-CoV-2, can be determined by biolayer interferometry, e.g. as described in Seydoux et al. (2020) https://doi.org/10.1101/2020.05.12.091298.

In one embodiment, the fragment of human ACE2 consists of 360 to 723 contiguous amino acids within the sequence according to SEQ ID NO: 1. Preferably, the fragment of human ACE2 consists of 380 to 723, 400 to 723, 420 to 723, 440 to 723, 460 to 723, 480 to 723 or 500 to 723 contiguous amino acids within the sequence according to SEQ ID NO: 1. Preferably, the fragment of human ACE2 consists of 520 to 723, 540 to 723, 560 to 723, 580 to 723 or 600 to 723 contiguous amino acids within the sequence according to SEQ ID NO: 1. More preferably, the fragment of human ACE2 consists of 620 to 723, 640 to 723, 660 to 723, 680 to 723, 700 to 723 or 720 to 723 contiguous amino acids within the sequence according to SEQ ID NO: 1.

In one embodiment, the fragment of human ACE2 comprises the amino acid residues K31 and K353, the numbering referring to SEQ ID NO: 1. In one embodiment, the fragment of human ACE2 comprises the amino acid residues Q24, D30, E35 and Q42, the numbering referring to SEQ ID NO:1. In one embodiment, the fragment of human ACE2 comprises the amino acid residues Q24, D30, K31, E35, Q42 and K353, the numbering referring to SEQ ID NO:1.

In one embodiment, the fragment of human ACE2 consists of 360 to 723 contiguous amino acids within the sequence according to SEQ ID NO: 1 and comprises the amino acid residues K31 and K353, the numbering referring to SEQ ID NO: 1. Preferably, the fragment of human ACE2 consists of 380 to 723, 400 to 723, 420 to 723, 440 to 723, 460 to 723, 480 to 723 or 500 to 723 contiguous amino acids within the sequence according to SEQ ID NO: 1 and comprises the amino acid residues K31 and K353, the numbering referring to SEQ ID NO: 1. Preferably, the fragment of human ACE2 consists of 520 to 723, 540 to 723, 560 to 723, 580 to 723 or 600 to 723 contiguous amino acids within the sequence according to SEQ ID NO: 1 and comprises the amino acid residues K31 and K353, the numbering referring to SEQ ID NO: 1. More preferably, the fragment of human ACE2 consists of 620 to 723, 640 to 723, 660 to 723, 680 to 723, 700 to 723 or 720 to 723 contiguous amino acids within the sequence according to SEQ ID NO: 1 and comprises the amino acid residues K31 and K353, the numbering referring to SEQ ID NO: 1.

In one embodiment, the fragment of human ACE2 consists of 360 to 723 contiguous amino acids within the sequence according to SEQ ID NO: 1 and comprises the amino acid residues Q24, D30, E35 and Q42, the numbering referring to SEQ ID NO: 1. Preferably, the fragment of human ACE2 consists of 380 to 723, 400 to 723, 420 to 723, 440 to 723, 460 to 723, 480 to 723 or 500 to 723 contiguous amino acids within the sequence according to SEQ ID NO: 1 and comprises the amino acid residues Q24, D30, E35 and Q42, the numbering referring to SEQ ID NO: 1. Preferably, the fragment of human ACE2 consists of 520 to 723, 540 to 723, 560 to 723, 580 to 723 or 600 to 723 contiguous amino acids within the sequence according to SEQ ID NO: 1 and comprises the amino acid residues Q24, D30, E35 and Q42, the numbering referring to SEQ ID NO: 1. More preferably, the fragment of human ACE2 consists of 620 to 723, 640 to 723, 660 to 723, 680 to 723, 700 to 723 or 720 to 723 contiguous amino acids within the sequence according to SEQ ID NO: 1 and comprises the amino acid residues Q24, D30, E35 and Q42, the numbering referring to SEQ ID NO: 1.

In one embodiment, the fragment of human ACE2 consists of 360 to 723 contiguous amino acids within the sequence according to SEQ ID NO: 1 and comprises the amino acid residues Q24, D30, K31, E35, Q42 and K353, the numbering referring to SEQ ID NO: 1. Preferably, the fragment of human ACE2 consists of 380 to 723, 400 to 723, 420 to 723, 440 to 723, 460 to 723, 480 to 723 or 500 to 723 contiguous amino acids within the sequence according to SEQ ID NO: 1 and comprises the amino acid residues Q24, D30, K31, E35, Q42 and K353, the numbering referring to SEQ ID NO: 1. Preferably, the fragment of human ACE2 consists of 520 to 723, 540 to 723, 560 to 723, 580 to 723 or 600 to 723 contiguous amino acids within the sequence according to SEQ ID NO: 1 and comprises the amino acid residues Q24, D30, K31, E35, Q42 and K353, the numbering referring to SEQ ID NO: 1. More preferably, the fragment of human ACE2 consists of 620 to 723, 640 to 723, 660 to 723, 680 to 723, 700 to 723 or 720 to 723 contiguous amino acids within the sequence according to SEQ ID NO: 1 and comprises the amino acid residues Q24, D30, K31, E35, Q42 and K353, the numbering referring to SEQ ID NO: 1.

In one embodiment, the fragment of human ACE2 consists of amino acids 18 to 380, 18 to 400, 18 to 420, 18 to 440, 18 to 460, 18 to 480 or 18 to 500 of the sequence according to SEQ ID NO: 1. Preferably, the fragment of human ACE2 consists of amino acids 18 to 520, 18 to 540, 18 to 560, 18 to 580 or 18 to 600 of the sequence according to SEQ ID NO: 1. More preferably, the fragment of human ACE2 consists of amino acids 18 to 605, 18 to 615, 18 to 620, 18 to 640, 18 to 660, 18 to 680 or 18 to 700 of the sequence according to SEQ ID NO: 1. Even more preferably, the fragment of human ACE2 consists of amino acids 18 to 710, 18 to 720 or 18 to 730 of the sequence according to SEQ ID NO: 1.

In one embodiment, the fragment of human ACE2 consists of the amino acid sequence according to SEQ ID NO:2. The amino acid sequence according to SEQ ID NO: 2 starts at amino acid Q18 and ends with amino acid G732 in the sequence according to SEQ ID NO: 1. The amino acid glycine at the C-terminal end of this fragment provides a high rotational freedom which favours the fusion of the two protein parts and increases the stability of the fusion protein. Additionally, the use of an ACE2 fragment comprising the amino acid sequence which starts at amino acid Q18 and ends with amino acid G732 in the sequence according to SEQ ID NO: 1 provides a better yield than a longer ACE2 fragment.

In one embodiment, the fragment of human ACE2 consists of the complete extracellular domain of human ACE2 which has the amino acid sequence according to SEQ ID NO:3.

In one embodiment, the fragment of human ACE2 consists of the amino acid sequence according to SEQ ID NO:12. The amino acid sequence according to SEQ ID NO: 12 starts at amino acid Q18 and ends with amino acid G605 in the sequence according to SEQ ID NO: 1.

In one embodiment, the fragment of human ACE2 is N-glycosylated at at least one amino acid residue selected from N53, N90, N103, N322, N432, N546 and N690, the numbering referring to SEQ ID NO: 1. In one embodiment, the fragment of human ACE2 is N-glycosylated at amino acid residues N53, N90 and, N322, the numbering referring to SEQ ID NO: 1. In one embodiment, the fragment of human ACE2 is N-glycosylated at amino acid residues N53, N90, N103, N322, N432, N546 and N690, the numbering referring to SEQ ID NO: 1.

In one embodiment, the fragment of human ACE2 consists of the amino acid sequence according to SEQ ID NO:2 and is N-glycosylated at at least one amino acid residue selected from N53, N90, N103, N322, N432, N546 and N690, the numbering referring to SEQ ID NO: 1. In one embodiment, the fragment of human ACE2 consists of the amino acid sequence according to SEQ ID NO:2 and is N-glycosylated at amino acid residues N53, N90 and N322, the numbering referring to SEQ ID NO: 1. In one embodiment, the fragment of human ACE2 consists of the amino acid sequence according to SEQ ID NO:2 and is N-glycosylated at amino acid residues N53, N90, N103, N322, N432, N546 and N690, the numbering referring to SEQ ID NO: 1.

In one embodiment, the fragment of human ACE2 consists of the amino acid sequence according to SEQ ID NO:3 and is N-glycosylated at at least one amino acid residue selected from N53, N90, N103, N322, N432, N546 and N690, the numbering referring to SEQ ID NO: 1. In one embodiment, the fragment of human ACE2 consists of the amino acid sequence according to SEQ ID NO:3 and is N-glycosylated at amino acid residues N53, N90 and N322, the numbering referring to SEQ ID NO: 1. In one embodiment, the fragment of human ACE2 consists of the amino acid sequence according to SEQ ID NO:3 and is N-glycosylated at amino acid residues N53, N90, N103, N322, N432, N546 and N690, the numbering referring to SEQ ID NO: 1.

In one embodiment, the fragment of human ACE2 comprises or is identified by 360 to 723 contiguous amino acids within the sequence according to SEQ ID NO: 1. Preferably, the fragment of human ACE2 comprises or is identified by 380 to 723, 400 to 723, 420 to 723, 440 to 723, 460 to 723, 480 to 723 or 500 to 723 contiguous amino acids within the sequence according to SEQ ID NO: 1. Preferably, the fragment of human ACE2 comprises or is identified by 520 to 723, 540 to 723, 560 to 723, 580 to 723 or 600 to 723 contiguous amino acids within the sequence according to SEQ ID NO: 1. More preferably, the fragment of human ACE2 comprises or is identified by 620 to 723, 640 to 723, 660 to 723, 680 to 723, 700 to 723 or 720 to 723 contiguous amino acids within the sequence according to SEQ ID NO: 1.

In one embodiment, the fragment of human ACE2 comprises the amino acid residues K31 and K353, the numbering referring to SEQ ID NO: 1. In one embodiment, the fragment of human ACE2 comprises the amino acid residues Q24, D30, E35 and Q42, the numbering referring to SEQ ID NO:1. In one embodiment, the fragment of human ACE2 comprises the amino acid residues Q24, D30, K31, E35, Q42 and K353, the numbering referring to SEQ ID NO:1.

In one embodiment, the fragment of human ACE2 comprises or is identified by 360 to 723 contiguous amino acids within the sequence according to SEQ ID NO: 1 and comprises the amino acid residues K31 and K353, the numbering referring to SEQ ID NO: 1. Preferably, the fragment of human ACE2 comprises or is identified by 380 to 723, 400 to 723, 420 to 723, 440 to 723, 460 to 723, 480 to 723 or 500 to 723 contiguous amino acids within the sequence according to SEQ ID NO: 1 and comprises the amino acid residues K31 and K353, the numbering referring to SEQ ID NO: 1. Preferably, the fragment of human ACE2 comprises or is identified by 520 to 723, 540 to 723, 560 to 723, 580 to 723 or 600 to 723 contiguous amino acids within the sequence according to SEQ ID NO: 1 and comprises the amino acid residues K31 and K353, the numbering referring to SEQ ID NO: 1. More preferably, the fragment of human ACE2 comprises or is identified by 620 to 723, 640 to 723, 660 to 723, 680 to 723, 700 to 723 or 720 to 723 contiguous amino acids within the sequence according to SEQ ID NO: 1 and comprises the amino acid residues K31 and K353, the numbering referring to SEQ ID NO: 1.

In one embodiment, the fragment of human ACE2 comprises or is identified by 360 to 723 contiguous amino acids within the sequence according to SEQ ID NO: 1 and comprises the amino acid residues Q24, D30, E35 and Q42, the numbering referring to SEQ ID NO: 1. Preferably, the fragment of human ACE2 comprises or is identified by 380 to 723, 400 to 723, 420 to 723, 440 to 723, 460 to 723, 480 to 723 or 500 to 723 contiguous amino acids within the sequence according to SEQ ID NO: 1 and comprises the amino acid residues Q24, D30, E35 and Q42, the numbering referring to SEQ ID NO: 1. Preferably, the fragment of human ACE2 comprises or is identified by 520 to 723, 540 to 723, 560 to 723, 580 to 723 or 600 to 723 contiguous amino acids within the sequence according to SEQ ID NO: 1 and comprises the amino acid residues Q24, D30, E35 and Q42, the numbering referring to SEQ ID NO: 1. More preferably, the fragment of human ACE2 comprises or is identified by 620 to 723, 640 to 723, 660 to 723, 680 to 723, 700 to 723 or 720 to 723 contiguous amino acids within the sequence according to SEQ ID NO: 1 and comprises the amino acid residues Q24, D30, E35 and Q42, the numbering referring to SEQ ID NO: 1.

In one embodiment, the fragment of human ACE2 comprises or is identified by 360 to 723 contiguous amino acids within the sequence according to SEQ ID NO: 1 and comprises the amino acid residues Q24, D30, K31, E35, Q42 and K353, the numbering referring to SEQ ID NO: 1. Preferably, the fragment of human ACE2 comprises or is identified by 380 to 723, 400 to 723, 420 to 723, 440 to 723, 460 to 723, 480 to 723 or 500 to 723 contiguous amino acids within the sequence according to SEQ ID NO: 1 and comprises the amino acid residues Q24, D30, K31, E35, Q42 and K353, the numbering referring to SEQ ID NO: 1. Preferably, the fragment of human ACE2 comprises or is identified by 520 to 723, 540 to 723, 560 to 723, 580 to 723 or 600 to 723 contiguous amino acids within the sequence according to SEQ ID NO: 1 and comprises the amino acid residues Q24, D30, K31, E35, Q42 and K353, the numbering referring to SEQ ID NO: 1. More preferably, the fragment of human ACE2 comprises or is identified by 620 to 723, 640 to 723, 660 to 723, 680 to 723, 700 to 723 or 720 to 723 contiguous amino acids within the sequence according to SEQ ID NO: 1 and comprises the amino acid residues Q24, D30, K31, E35, Q42 and K353, the numbering referring to SEQ ID NO: 1.

In one embodiment, the fragment of human ACE2 comprises or is identified by amino acids 18 to 380, 18 to 400, 18 to 420, 18 to 440, 18 to 460, 18 to 480 or 18 to 500 of the sequence according to SEQ ID NO: 1. Preferably, the fragment of human ACE2 comprises or is identified by amino acids 18 to 520, 18 to 540, 18 to 560, 18 to 580 or 18 to 600 of the sequence according to SEQ ID NO: 1. More preferably, the fragment of human ACE2 comprises or is identified by amino acids 18 to 605, 18 to 615, 18 to 620, 18 to 640, 18 to 660, 18 to 680 or 18 to 700 of the sequence according to SEQ ID NO: 1. Even more preferably, the fragment of human ACE2 comprises or is identified by amino acids 18 to 710, 18 to 720 or 18 to 730 of the sequence according to SEQ ID NO: 1.

In one embodiment, the fragment of human ACE2 comprises or is identified by the amino acid sequence according to SEQ ID NO:2. The amino acid sequence according to SEQ ID NO: 2 starts at amino acid Q18 and ends with amino acid G732 in the sequence according to SEQ ID NO: 1. The amino acid glycine at the C-terminal end of this fragment provides a high rotational freedom which favours the fusion of the two protein parts and increases the stability of the fusion protein. Additionally, the use of an ACE2 fragment comprising or which is identified by the amino acid sequence which starts at amino acid Q18 and ends with amino acid G732 in the sequence according to SEQ ID NO: 1 provides a better yield than a longer ACE2 fragment.

In one embodiment, the fragment of human ACE2 comprises or is identified by the complete extracellular domain of human ACE2 which has the amino acid sequence according to SEQ ID NO:3.

In one embodiment, the fragment of human ACE2 comprises or is identified by the amino acid sequence according to SEQ ID NO:12. The amino acid sequence according to SEQ ID NO: 12 starts at amino acid Q18 and ends with amino acid G605 in the sequence according to SEQ ID NO: 1.

In one embodiment, the fragment of human ACE2 is N-glycosylated at at least one amino acid residue selected from N53, N90, N103, N322, N432, N546 and N690, the numbering referring to SEQ ID NO: 1. In one embodiment, the fragment of human ACE2 is N-glycosylated at amino acid residues N53, N90 and, N322, the numbering referring to SEQ ID NO: 1. In one embodiment, the fragment of human ACE2 is N-glycosylated at amino acid residues N53, N90, N103, N322, N432, N546 and N690, the numbering referring to SEQ ID NO: 1.

In one embodiment, the fragment of human ACE2 comprises or is identified by the amino acid sequence according to SEQ ID NO:2 and is N-glycosylated at at least one amino acid residue selected from N53, N90, N103, N322, N432, N546 and N690, the numbering referring to SEQ ID NO: 1. In one embodiment, the fragment of human ACE2 comprises or is identified by of the amino acid sequence according to SEQ ID NO:2 and is N-glycosylated at amino acid residues N53, N90 and N322, the numbering referring to SEQ ID NO: 1. In one embodiment, the fragment of human ACE2 comprises or is identified by the amino acid sequence according to SEQ ID NO:2 and is N-glycosylated at amino acid residues N53, N90, N103, N322, N432, N546 and N690, the numbering referring to SEQ ID NO: 1.

In one embodiment, the fragment of human ACE2 comprises or is identified by the amino acid sequence according to SEQ ID NO:3 and is N-glycosylated at at least one amino acid residue selected from N53, N90, N103, N322, N432, N546 and N690, the numbering referring to SEQ ID NO: 1. In one embodiment, the fragment of human ACE2 comprises or is identified by the amino acid sequence according to SEQ ID NO:3 and is N-glycosylated at amino acid residues N53, N90 and N322, the numbering referring to SEQ ID NO: 1. In one embodiment, the fragment of human ACE2 comprises or is identified by the amino acid sequence according to SEQ ID NO:3 and is N-glycosylated at amino acid residues N53, N90, N103, N322, N432, N546 and N690, the numbering referring to SEQ ID NO: 1.

The term “N-glycosylated” or “N-glycosylation” means that a glycan structure is attached to the amide nitrogen of an asparagine residue of a protein. A glycan is a branched, flexible chain of carbohydrates and the exact structure of the glycan attached to the asparagine residue of a protein depends on the expression system used for glycoprotein production.

A “variant” of the fragment of human ACE2 refers to a fragment as defined above, wherein compared to the corresponding sequence in the amino acid sequence of wild-type, full-length human ACE2 according to SEQ ID NO: 1 at least one amino acid residue is different or at least two, at least three, at least four, at least five, at least six, at least seven, at least eight, at least nine, at least ten, at least eleven or at least thirteen amino acids are different. The “variant” of the fragment of human ACE2 comprises one or more amino acid substitutions in the sequence of the fragment of human ACE2. A “variant” of the fragment of human ACE2 does not comprise any amino acid additions or deletions compared to the sequence from which the variant is derived. In one embodiment, the variant the fragment of human ACE2 is a variant of the fragment of human ACE2 according to SEQ ID NO: 2 or 3 and does not comprise any amino acid additions or deletions compared to the sequence according to SEQ ID NO: 2 or 3, i.e. it has the same length as the sequence according to SEQ ID NO: 2 or 3. Within the scope of the present invention a variant of the fragment of human ACE2 is capable of binding to the S protein of at least one coronavirus, in particular to the S protein of SARS-CoV-2. The binding of a variant of the fragment of human ACE2 to the S protein of at least one coronavirus, in particular to the S protein of SARS-CoV-2, can be determined as described above for fragments of human ACE2.

In one embodiment, the variant of the fragment of human ACE2 differs from the corresponding amino acid sequence in the sequence according to SEQ ID NO: 1 by one amino acid. In another embodiment, the variant of the fragment of human ACE2 differs from the corresponding amino acid sequence in the sequence according to SEQ ID NO: 1 by two amino acids. In still another embodiment, the variant of the fragment of human ACE2 differs from the corresponding amino acid sequence in the sequence according to SEQ ID NO: 1 by three amino acids. In still another embodiment, the variant of the fragment of human ACE2 differs from the corresponding amino acid sequence in the sequence according to SEQ ID NO: 1 by four amino acids. In still another embodiment, the variant of the fragment of human ACE2 differs from the corresponding amino acid sequence in the sequence according to SEQ ID NO: 1 by five amino acids. In still another embodiment, the variant of the fragment of human ACE2 differs from the corresponding amino acid sequence in the sequence according to SEQ ID NO: 1 by six amino acids. In still another embodiment, the variant of the fragment of human ACE2 differs from the corresponding amino acid sequence in the sequence according to SEQ ID NO: 1 by seven amino acids. In still another embodiment, the variant of the fragment of human ACE2 differs from the corresponding amino acid sequence in the sequence according to SEQ ID NO: 1 by eight amino acids. In still another embodiment, the variant of the fragment of human ACE2 differs from the corresponding amino acid sequence in the sequence according to SEQ ID NO: 1 by nine amino acids. In still another embodiment, the variant of the fragment of human ACE2 differs from the corresponding amino acid sequence in the sequence according to SEQ ID NO: 1 by ten amino acids. In still another embodiment, the variant of the fragment of human ACE2 differs from the corresponding amino acid sequence in the sequence according to SEQ ID NO: 1 by eleven amino acids. In still another embodiment, the variant of the fragment of human ACE2 differs from the corresponding amino acid sequence in the sequence according to SEQ ID NO: 1 by twelve amino acids. In still another embodiment, the variant of the fragment of human ACE2 differs from the corresponding amino acid sequence in the sequence according to SEQ ID NO: 1 by thirteen amino acids.

One variant of the fragment of human ACE2 may be an enzymatically inactive variant. “The enzymatically inactive variant of the fragment of human ACE2” lacks the ability to cleave angiotensin II to Ang1-7. The enzymatic activity of human ACE2 can be determined by methods known to the skilled person. Suitable kits for determining the enzymatic activity of human ACE2 are commercially available, for example from the companies BioVision or Anaspec. By using an enzymatically inactive ACE2 variant any side effects associated with the enzymatic activity of ACE2 such as effects on the cardiovascular system or the regulation of blood pressure are eliminated. Further, the risk of counterbalancing the RAS-MAS equilibrium is reduced.

The enzymatically inactive variant of the fragment of human ACE2 may comprise one or more mutations of amino acids within the catalytic centre of ACE2. In particular, the enzymatically inactive variant of the fragment of human ACE2 comprises a mutation of the wildtype histidine at residue 374 of the sequence according to SEQ ID NO: 1 and/or a mutation of the wildtype histidine at residue 378 of the sequence according to SEQ ID NO: 1. The wild-type histidine may be mutated to any amino acid other than histidine and particularly, the wild-type histidine is mutated to asparagine. Preferably, the enzymatically inactive variant of the fragment of human ACE2 comprises a H374N and a H378N mutation, the numbering referring to the sequence according to SEQ ID NO: 1.

In another embodiment, the enzymatically inactive variant of the fragment of human ACE2 comprises a mutation at one or more of the following amino acid residues, the numbering referring to the sequence according to SEQ ID NO: 1: residue 345 (histidine in wild-type), 273 (arginine in wild-type), 402 (glutamic acid in wild-type) and 505 (histidine in wild-type). In one embodiment, the enzymatically inactive variant of the fragment of human ACE2 comprises a mutation of histidine at residue 345 to alanine or leucine, a mutation of arginine at residue 273 to alanine, glutamine or lysine, a mutation of glutamic acid at residue 402 to alanine and/or a mutation of histidine at residue 505 to alanine or leucine, the numbering referring to the sequence according to SEQ ID NO: 1.

In one embodiment, the fragment of human ACE2 consists of amino acids 18 to 380, 18 to 400, 18 to 420, 18 to 440, 18 to 460, 18 to 480 or 18 to 500 of the sequence according to SEQ ID NO: 1 and comprises a H374N and a H378N mutation, the numbering referring to the sequence according to SEQ ID NO: 1. Preferably, the fragment of human ACE2 consists of amino acids 18 to 520, 18 to 540, 18 to 560, 18 to 580 or 18 to 600 of the sequence according to SEQ ID NO: 1 and comprises a H374N and a H378N mutation, the numbering referring to the sequence according to SEQ ID NO: 1. More preferably, the fragment of human ACE2 consists of amino acids 18 to 615, 18 to 620, 18 to 640, 18 to 660, 18 to 680 or 18 to 700 of the sequence according to SEQ ID NO: 1 and comprises a H374N and a H378N mutation, the numbering referring to the sequence according to SEQ ID NO: 1. Even more preferably, the fragment of human ACE2 consists of amino acids 18 to 710, 18 to 720 or 18 to 730 of the sequence according to SEQ ID NO: 1 and comprises a H374N and a H378N mutation, the numbering referring to the sequence according to SEQ ID NO: 1.

In one embodiment, the fragment of human ACE2 consists of the sequence according to SEQ ID NO: 2 and comprises a H374N and a H378N mutation, the numbering referring to the sequence according to SEQ ID NO: 1. In one embodiment, the fragment of human ACE2 consists of the sequence according to SEQ ID NO: 3 and comprises a H374N and a H378N mutation, the numbering referring to the sequence according to SEQ ID NO: 1.

In one embodiment, the fragment of human ACE2 comprises or is identified by amino acids 18 to 380, 18 to 400, 18 to 420, 18 to 440, 18 to 460, 18 to 480 or 18 to 500 of the sequence according to SEQ ID NO: 1 and comprises a H374N and a H378N mutation, the numbering referring to the sequence according to SEQ ID NO: 1. Preferably, the fragment of human ACE2 comprises or is identified by amino acids 18 to 520, 18 to 540, 18 to 560, 18 to 580 or 18 to 600 of the sequence according to SEQ ID NO: 1 and comprises a H374N and a H378N mutation, the numbering referring to the sequence according to SEQ ID NO: 1. More preferably, the fragment of human ACE2 comprises or is identified by amino acids 18 to 615, 18 to 620, 18 to 640, 18 to 660, 18 to 680 or 18 to 700 of the sequence according to SEQ ID NO: 1 and comprises a H374N and a H378N mutation, the numbering referring to the sequence according to SEQ ID NO: 1. Even more preferably, the fragment of human ACE2 comprises or is identified by amino acids 18 to 710, 18 to 720 or 18 to 730 of the sequence according to SEQ ID NO: 1 and comprises a H374N and a H378N mutation, the numbering referring to the sequence according to SEQ ID NO: 1.

In one embodiment, the fragment of human ACE2 comprises or is identified by the sequence according to SEQ ID NO: 2 and comprises a H374N and a H378N mutation, the numbering referring to the sequence according to SEQ ID NO: 1. In one embodiment, the fragment of human ACE2 comprises or is identified by the sequence according to SEQ ID NO: 3 and comprises a H374N and a H378N mutation, the numbering referring to the sequence according to SEQ ID NO: 1.

Another variant of the fragment of human ACE2 may be a variant which inhibits shedding of ACE2. It was shown that ACE2 is shed from human airway epithelia by cleavage of the ACE2 ectodomain and that ADAM17 regulates ACE2 cleavage. Further, a point mutation at leucine 584 of full-length ACE2 which is located in the ectodomain of ACE2 abolished shedding (Jia et al. (2009) Am. J. Physiol. Lung Cell. Mol. Physiol. 297(1): L84-96). Hence, in one embodiment the variant of the fragment of human ACE2 comprises a mutation at leucine 584, the numbering referring to the sequence according to SEQ ID NO:1. In one embodiment the mutation at leucine 584 is a L584A mutation.

In one embodiment, the fragment of human ACE2 consists of the sequence according to SEQ ID NO: 2 and comprises a L584A mutation, the numbering referring to the sequence according to SEQ ID NO: 1.

In one embodiment, the fragment of human ACE2 consists of the sequence according to SEQ ID NO: 3 and comprises a L584A mutation, the numbering referring to the sequence according to SEQ ID NO: 1.

In one embodiment, the fragment of human ACE2 comprises or is identified by the sequence according to SEQ ID NO: 2 and comprises a L584A mutation, the numbering referring to the sequence according to SEQ ID NO: 1.

In one embodiment, the fragment of human ACE2 comprises or is identified by the sequence according to SEQ ID NO: 3 and comprises a L584A mutation, the numbering referring to the sequence according to SEQ ID NO: 1.

In one embodiment, the variant of the fragment of human ACE2 comprises a H374N mutation, a H378N mutation and a L584A mutation, the numbering referring to the sequence according to SEQ ID NO: 1.

In one embodiment, the fragment of human ACE2 consists of the sequence according to SEQ ID NO: 2 and comprises a H374N mutation, a H378N mutation and a L584A mutation, the numbering referring to the sequence according to SEQ ID NO: 1.

In one embodiment, the fragment of human ACE2 consists of the sequence according to SEQ ID NO: 3 and comprises a H374N mutation, a H378N mutation and a L584A mutation, the numbering referring to the sequence according to SEQ ID NO: 1.

In one embodiment, the fragment of human ACE2 comprises or is identified by the sequence according to SEQ ID NO: 2 and comprises a H374N mutation, a H378N mutation and a L584A mutation, the numbering referring to the sequence according to SEQ ID NO: 1.

In one embodiment, the fragment of human ACE2 comprises or is identified by the sequence according to SEQ ID NO: 3 and comprises a H374N mutation, a H378N mutation and a L584A mutation, the numbering referring to the sequence according to SEQ ID NO: 1.

Another variant of the fragment of human ACE2 may be a variant which inhibits cleavage of ACE2 by the protease TMPRSS2. It was shown that ACE2 proteolysis by TMPRSS2 augments entry of SARS-CoV (Heurich et al. (2014) J. Virol. 88(2): 1293-1307). TMPRSS2 also plays a role in the entry of SARS-CoV-2 into the cells (Hoffmann et al. (2020) Cell 181: 1-10). To abolish cleavage of ACE2 by TMPRSS2, the amino acid residues essential for the cleavage may be mutated. It was shown that arginine and lysine residues within the amino acid region spanning amino acids 697 to 716 of ACE2 are essential for ACE2 cleavage by TMPRSS2 (Heurich et al. (2014) J. Virol. 88(2): 1293-1307). Hence, in one embodiment the variant of the fragment of human ACE2 comprises a mutation at at least one residue selected from amino acids 697, 702, 705, 708, 710 and 716, the numbering referring to SEQ ID NO: 1. Preferably, the variant of the fragment of human ACE2 comprises a mutation at at least two or three residues selected from amino acids 697, 702, 705, 708, 710 and 716, the numbering referring to SEQ ID NO: 1. More preferably, the variant of the fragment of human ACE2 comprises a mutation at at least four or five residues selected from amino acids 697, 702, 705, 708, 710 and 716, the numbering referring to SEQ ID NO: 1. Most preferably, the variant of the fragment of human ACE2 comprises a mutation at residues 697, 702, 705, 708, 710 and 716, the numbering referring to SEQ ID NO: 1. The wild-type amino acid residue at any of these residues may be mutated to any other amino acid and particularly, the wild-type amino acid residue is mutated to alanine.

In one embodiment, the variant of the fragment of human ACE2 comprises at least one of the following mutations: R697A, K702A, R705A, R708A, R710A and R716A, the numbering referring to SEQ ID NO: 1. Preferably, the variant of the fragment of human ACE2 comprises at least two or three of the following mutations: R697A, K702A, R705A, R708A, R710A and R716A, the numbering referring to SEQ ID NO: 1. More preferably, the variant of the fragment of human ACE2 comprises at least four or five of the following mutations: R697A, K702A, R705A, R708A, R710A and R716A, the numbering referring to SEQ ID NO: 1. Most preferably, the variant of the fragment of human ACE2 comprises the following mutations: R697A, K702A, R705A, R708A, R710A and R716A, the numbering referring to SEQ ID NO: 1.

The variant of the fragment of human ACE2 may further comprise mutations at residues 619, 621 and/or 625, the numbering referring to SEQ ID NO: 1. In particular, the variant of the fragment of human ACE2 may further comprise the following mutations: K619A, R621A and/or K625A, the numbering referring to SEQ ID NO: 1.

Hence, in one embodiment, the variant of the fragment of human ACE2 comprises the following mutations: K619A, R621A, K625A, R697A, K702A, R705A, R708A, R710A and R716A, the numbering referring to SEQ ID NO: 1.

In one embodiment, the fragment of human ACE2 consists of the sequence according to SEQ ID NO: 2 and comprises a mutation at at least one residue selected from amino acids 697, 702, 705, 708, 710 and 716, the numbering referring to SEQ ID NO: 1. In one embodiment, the fragment of human ACE2 consists of the sequence according to SEQ ID NO: 2 and comprises a mutation at residues 697, 702, 705, 708, 710 and 716, the numbering referring to SEQ ID NO: 1. In one embodiment, the fragment of human ACE2 consists of the sequence according to SEQ ID NO: 2 and comprises at least one of the following mutations: R697A, K702A, R705A, R708A, R710A and R716A, the numbering referring to SEQ ID NO: 1. In one embodiment, the fragment of human ACE2 consists of the sequence according to SEQ ID NO: 2 and comprises the following mutations: R697A, K702A, R705A, R708A, R710A and R716A, the numbering referring to SEQ ID NO: 1.

In one embodiment, the fragment of human ACE2 consists of the sequence according to SEQ ID NO: 3 and comprises a mutation at at least one residue selected from amino acids 697, 702, 705, 708, 710 and 716, the numbering referring to SEQ ID NO: 1. In one embodiment, the fragment of human ACE2 consists of the sequence according to SEQ ID NO: 3 and comprises a mutation at residues 697, 702, 705, 708, 710 and 716, the numbering referring to SEQ ID NO: 1. In one embodiment, the fragment of human ACE2 consists of the sequence according to SEQ ID NO: 3 and comprises at least one of the following mutations: R697A, K702A, R705A, R708A, R710A and R716A, the numbering referring to SEQ ID NO: 1. In one embodiment, the fragment of human ACE2 consists of the sequence according to SEQ ID NO: 3 and comprises the following mutations: R697A, K702A, R705A, R708A, R710A and R716A, the numbering referring to SEQ ID NO: 1.

In one embodiment, the fragment of human ACE2 consists of the sequence according to SEQ ID NO: 2 and comprises a H374N mutation, a H378N mutation, a L584A mutation and the following mutations: R697A, K702A, R705A, R708A, R710A and R716A, the numbering referring to the sequence according to SEQ ID NO: 1.

In one embodiment, the fragment of human ACE2 consists of the sequence according to SEQ ID NO: 3 and comprises a H374N mutation, a H378N mutation, a L584A mutation and the following mutations: R697A, K702A, R705A, R708A, R710A and R716A, the numbering referring to the sequence according to SEQ ID NO: 1.

In one embodiment, the fragment of human ACE2 consists of the sequence according to SEQ ID NO: 2 and comprises a mutation at at least one residue selected from amino acids 619, 621, 625, 697, 702, 705, 708, 710 and 716, the numbering referring to SEQ ID NO: 1. In one embodiment, the fragment of human ACE2 consists of the sequence according to SEQ ID NO: 2 and comprises a mutation at residues 619, 621, 625, 697, 702, 705, 708, 710 and 716, the numbering referring to SEQ ID NO: 1. In one embodiment, the fragment of human ACE2 consists of the sequence according to SEQ ID NO: 2 and comprises at least one of the following mutations: K619A, R621A, K625A, R697A, K702A, R705A, R708A, R710A and R716A, the numbering referring to SEQ ID NO: 1. In one embodiment, the fragment of human ACE2 consists of the sequence according to SEQ ID NO: 2 and comprises the following mutations: K619A, R621A, K625A, R697A, K702A, R705A, R708A, R710A and R716A, the numbering referring to SEQ ID NO: 1.

In one embodiment, the fragment of human ACE2 consists of the sequence according to SEQ ID NO: 3 and comprises a mutation at at least one residue selected from amino acids 619, 621, 625, 697, 702, 705, 708, 710 and 716, the numbering referring to SEQ ID NO: 1. In one embodiment, the fragment of human ACE2 consists of the sequence according to SEQ ID NO: 3 and comprises a mutation at residues 619, 621, 625, 697, 702, 705, 708, 710 and 716, the numbering referring to SEQ ID NO: 1. In one embodiment, the fragment of human ACE2 consists of the sequence according to SEQ ID NO: 3 and comprises at least one of the following mutations: K619A, R621A, K625A, R697A, K702A, R705A, R708A, R710A and R716A, the numbering referring to SEQ ID NO: 1. In one embodiment, the fragment of human ACE2 consists of the sequence according to SEQ ID NO: 3 and comprises the following mutations: K619A, R621A, K625A, R697A, K702A, R705A, R708A, R710A and R716A, the numbering referring to SEQ ID NO: 1.

In one embodiment, the fragment of human ACE2 consists of the sequence according to SEQ ID NO: 2 and comprises a H374N mutation, a H378N mutation, a L584A mutation and the following mutations: K619A, R621A, K625A, R697A, K702A, R705A, R708A, R710A and R716A, the numbering referring to the sequence according to SEQ ID NO: 1.

In one embodiment, the fragment of human ACE2 consists of the sequence according to SEQ ID NO: 3 and comprises a H374N mutation, a H378N mutation, a L584A mutation and the following mutations: K619A, R621A, K625A, R697A, K702A, R705A, R708A, R710A and R716A, the numbering referring to the sequence according to SEQ ID NO: 1.

In one embodiment, the fragment of human ACE2 comprises or is identified by the sequence according to SEQ ID NO: 2 and comprises a mutation at at least one residue selected from amino acids 697, 702, 705, 708, 710 and 716, the numbering referring to SEQ ID NO: 1. In one embodiment, the fragment of human ACE2 comprises or is identified by the sequence according to SEQ ID NO: 2 and comprises a mutation at residues 697, 702, 705, 708, 710 and 716, the numbering referring to SEQ ID NO: 1. In one embodiment, the fragment of human ACE2 comprises or is identified by the sequence according to SEQ ID NO: 2 and comprises at least one of the following mutations: R697A, K702A, R705A, R708A, R710A and R716A, the numbering referring to SEQ ID NO: 1. In one embodiment, the fragment of human ACE2 comprises or is identified by the sequence according to SEQ ID NO: 2 and comprises the following mutations: R697A, K702A, R705A, R708A, R710A and R716A, the numbering referring to SEQ ID NO: 1.

In one embodiment, the fragment of human ACE2 comprises or is identified by the sequence according to SEQ ID NO: 3 and comprises a mutation at at least one residue selected from amino acids 697, 702, 705, 708, 710 and 716, the numbering referring to SEQ ID NO: 1. In one embodiment, the fragment of human ACE2 comprises or is identified by the sequence according to SEQ ID NO: 3 and comprises a mutation at residues 697, 702, 705, 708, 710 and 716, the numbering referring to SEQ ID NO: 1. In one embodiment, the fragment of human ACE2 comprises or is identified by the sequence according to SEQ ID NO: 3 and comprises at least one of the following mutations: R697A, K702A, R705A, R708A, R710A and R716A, the numbering referring to SEQ ID NO: 1. In one embodiment, the fragment of human ACE2 comprises or is identified by the sequence according to SEQ ID NO: 3 and comprises the following mutations: R697A, K702A, R705A, R708A, R710A and R716A, the numbering referring to SEQ ID NO: 1.

In one embodiment, the fragment of human ACE2 comprises or is identified by the sequence according to SEQ ID NO: 2 and comprises a H374N mutation, a H378N mutation, a L584A mutation and the following mutations: R697A, K702A, R705A, R708A, R710A and R716A, the numbering referring to the sequence according to SEQ ID NO: 1.

In one embodiment, the fragment of human ACE2 comprises or is identified by the sequence according to SEQ ID NO: 3 and comprises a H374N mutation, a H378N mutation, a L584A mutation and the following mutations: R697A, K702A, R705A, R708A, R710A and R716A, the numbering referring to the sequence according to SEQ ID NO: 1.

In one embodiment, the fragment of human ACE2 comprises or is identified by the sequence according to SEQ ID NO: 2 and comprises a mutation at at least one residue selected from amino acids 619, 621, 625, 697, 702, 705, 708, 710 and 716, the numbering referring to SEQ ID NO: 1. In one embodiment, the fragment of human ACE2 comprises or is identified by the sequence according to SEQ ID NO: 2 and comprises a mutation at residues 619, 621, 625, 697, 702, 705, 708, 710 and 716, the numbering referring to SEQ ID NO: 1. In one embodiment, the fragment of human ACE2 comprises or is identified by the sequence according to SEQ ID NO: 2 and comprises at least one of the following mutations: K619A, R621A, K625A, R697A, K702A, R705A, R708A, R710A and R716A, the numbering referring to SEQ ID NO: 1. In one embodiment, the fragment of human ACE2 comprises or is identified by the sequence according to SEQ ID NO: 2 and comprises the following mutations: K619A, R621A, K625A, R697A, K702A, R705A, R708A, R710A and R716A, the numbering referring to SEQ ID NO: 1.

In one embodiment, the fragment of human ACE2 comprises or is identified by the sequence according to SEQ ID NO: 3 and comprises a mutation at at least one residue selected from amino acids 619, 621, 625, 697, 702, 705, 708, 710 and 716, the numbering referring to SEQ ID NO: 1. In one embodiment, the fragment of human ACE2 comprises or is identified by the sequence according to SEQ ID NO: 3 and comprises a mutation at residues 619, 621, 625, 697, 702, 705, 708, 710 and 716, the numbering referring to SEQ ID NO: 1. In one embodiment, the fragment of human ACE2 comprises or is identified by the sequence according to SEQ ID NO: 3 and comprises at least one of the following mutations: K619A, R621A, K625A, R697A, K702A, R705A, R708A, R710A and R716A, the numbering referring to SEQ ID NO: 1. In one embodiment, the fragment of human ACE2 comprises or is identified by the sequence according to SEQ ID NO: 3 and comprises the following mutations: K619A, R621A, K625A, R697A, K702A, R705A, R708A, R710A and R716A, the numbering referring to SEQ ID NO: 1.

In one embodiment, the fragment of human ACE2 comprises or is identified by the sequence according to SEQ ID NO: 2 and comprises a H374N mutation, a H378N mutation, a L584A mutation and the following mutations: K619A, R621A, K625A, R697A, K702A, R705A, R708A, R710A and R716A, the numbering referring to the sequence according to SEQ ID NO: 1.

In one embodiment, the fragment of human ACE2 comprises or is identified by the sequence according to SEQ ID NO: 3 and comprises a H374N mutation, a H378N mutation, a L584A mutation and the following mutations: K619A, R621A, K625A, R697A, K702A, R705A, R708A, R710A and R716A, the numbering referring to the sequence according to SEQ ID NO: 1.

Another variant of the fragment of human ACE2 may be a variant which provides an additional cysteine for the formation of disulfide bridges between two ACE2 molecules. The disulfide bridge increases the intrinsic stability of the fusion protein and may also have an effect on the binding of the fusion protein to its target. The additional cysteine may be provided by a substitution of serine 645 in the numbering of SEQ ID NO: 1 with cysteine.

Hence, in one embodiment, the variant of the fragment of human ACE2 comprises a S645C mutation, the numbering referring to SEQ ID NO: 1.

In one embodiment, the fragment of human ACE2 consists of the sequence according to SEQ ID NO: 2 and comprises a S645C mutation, the numbering referring to SEQ ID NO: 1.

In one embodiment, the fragment of human ACE2 consists of the sequence according to SEQ ID NO: 3 and comprises a S645C mutation, the numbering referring to SEQ ID NO: 1.

In one embodiment, the fragment of human ACE2 consists of the sequence according to SEQ ID NO: 2 and comprises a H374N mutation, a H378N mutation, a L584A mutation and a S645C mutation, the numbering referring to SEQ ID NO: 1.

In one embodiment, the fragment of human ACE2 consists of the sequence according to SEQ ID NO: 3 and comprises a H374N mutation, a H378N mutation, a L584A mutation and a S645C mutation, the numbering referring to SEQ ID NO: 1.

In one embodiment, the fragment of human ACE2 comprises or is identified by the sequence according to SEQ ID NO: 2 and comprises a S645C mutation, the numbering referring to SEQ ID NO: 1.

In one embodiment, the fragment of human ACE2 comprises or is identified by the sequence according to SEQ ID NO: 3 and comprises a S645C mutation, the numbering referring to SEQ ID NO: 1.

In one embodiment, the fragment of human ACE2 comprises or is identified by the sequence according to SEQ ID NO: 2 and comprises a H374N mutation, a H378N mutation, a L584A mutation and a S645C mutation, the numbering referring to SEQ ID NO: 1.

In one embodiment, the fragment of human ACE2 comprises or is identified by the sequence according to SEQ ID NO: 3 and comprises a H374N mutation, a H378N mutation, a L584A mutation and a S645C mutation, the numbering referring to SEQ ID NO: 1.

Another variant of the fragment of human ACE2 may be a variant which inhibits dimerization. Hence, in one embodiment the variant of the fragment of human ACE2 comprises a mutation at amino acid Q139, the numbering referring to SEQ ID NO: 1. In one embodiment the variant of the fragment of human ACE2 comprises a Q139A mutation, the numbering referring to SEQ ID NO: 1.

In one embodiment, the fragment of human ACE2 consists of the sequence according to SEQ ID NO: 2 and comprises a Q139A mutation, the numbering referring to SEQ ID NO: 1.

In one embodiment, the fragment of human ACE2 consists of the sequence according to SEQ ID NO: 3 and comprises a Q139A mutation, the numbering referring to SEQ ID NO: 1.

In one embodiment, the fragment of human ACE2 consists of the sequence according to SEQ ID NO: 2 and comprises a H374N mutation, a H378N mutation, a L584A mutation and a Q139A mutation, the numbering referring to SEQ ID NO: 1.

In one embodiment, the fragment of human ACE2 consists of the sequence according to SEQ ID NO: 3 and comprises a H374N mutation, a H378N mutation, a L584A mutation and a Q139A mutation, the numbering referring to SEQ ID NO: 1.

In one embodiment, the fragment of human ACE2 consists of the sequence according to SEQ ID NO: 12 and comprises a Q139A mutation, the numbering referring to SEQ ID NO: 1.

In one embodiment, the fragment of human ACE2 consists of the sequence according to SEQ ID NO: 12 and comprises a H374N mutation, a H378N mutation, a L584A mutation and a Q139A mutation, the numbering referring to SEQ ID NO: 1.

In one embodiment, the fragment of human ACE2 comprises or is identified by the sequence according to SEQ ID NO: 2 and comprises a Q139A mutation, the numbering referring to SEQ ID NO: 1.

In one embodiment, the fragment of human ACE2 comprises or is identified by the sequence according to SEQ ID NO: 3 and comprises a Q139A mutation, the numbering referring to SEQ ID NO: 1.

In one embodiment, the fragment of human ACE2 comprises or is identified by the sequence according to SEQ ID NO: 2 and comprises a H374N mutation, a H378N mutation, a L584A mutation and a Q139A mutation, the numbering referring to SEQ ID NO: 1.

In one embodiment, the fragment of human ACE2 comprises or is identified by the sequence according to SEQ ID NO: 3 and comprises a H374N mutation, a H378N mutation, a L584A mutation and a Q139A mutation, the numbering referring to SEQ ID NO: 1.

In one embodiment, the fragment of human ACE2 comprises or is identified by the sequence according to SEQ ID NO: 12 and comprises a Q139A mutation, the numbering referring to SEQ ID NO: 1.

In one embodiment, the fragment of human ACE2 comprises or is identified by the sequence according to SEQ ID NO: 12 and comprises a H374N mutation, a H378N mutation, a L584A mutation and a Q139A mutation, the numbering referring to SEQ ID NO: 1.

In one embodiment, the second part of the fusion protein of the present invention comprises the Fc portion of human IgM or human IgG2 or a fragment thereof.

In one embodiment, the second part of the fusion protein of the present invention comprises the Fc portion of human IgM. The full-length Fc portion of human IgM comprises the CH2, CH3, CH4 domains and the tailpiece of human IgM linked together to form the Fc portion. A full-length human IgM antibody additionally comprises the Fab fragment which comprises the heavy chain variable region and the CH1 domain. Preferably, the Fc portion of human IgM used in the fusion protein of the present invention has the sequence according to SEQ ID NO: 4. The fusion protein of the present invention forms homodimers containing two polypeptide chains with the same amino acid sequence, preferably the amino acid sequence according to SEQ ID NO: 4, which polypeptide chains are linked by disulfide bonds.

It is known that immunoglobulins of the IgM subclass exist as pentamers and hexamers of homodimers due to disulfide bonds between the Fc domain of the homodimers (Müller et al. (2013) Proc. Natl. Acad. Sci USA 110(25): 10183-10188), resulting in ten or twelve antigen-binding sites that allow them to potently neutralize antigens. Further, immunoglobulins of the IgM subclass do not bind to Fcγ receptors. Since it has been discussed that Fcγ receptors play a role in antibody-dependent enhancement of viral pathogenesis (see, e.g., Bournazos et al. (2020) Nature Reviews Immunology 20: 633-643), the antibody-dependent enhancement can be avoided by using a fusion protein comprising the Fc portion of human IgM or a variant or fragment of the Fc portion of human IgM.

“A variant of the Fc portion of human IgM” refers to the Fc portion of human IgM which has one or more amino acid substitutions compared to the wild-type Fc portion of human IgM according to SEQ ID NO: 4. In one embodiment, the variant of the Fc portion of human IgM has one to twelve, one to eleven, one to ten, one to nine, one to eight, one to seven, one to six, one to five, one to four, one to three, one or two amino acid substitutions compared to the wild-type Fc portion of human IgM according to SEQ ID NO: 4. In one embodiment, the variant of the Fc portion of human IgM has one, two, three, four, five, six, seven, eight, nine, ten, eleven or twelve amino acid substitutions compared to the wild-type Fc portion of human IgM according to SEQ ID NO: 4. The variant of the Fc region of human IgM is capable of forming multimers, preferably hexamers. The formation of multimers can be determined by size exclusion chromatography coupled to multi-angle light scattering (SEC-MALS) as described in the examples herein.

In one embodiment, the variant of the Fc portion of human IgM comprises a mutation at position 221 and/or 222, the numbering referring to SEQ ID NO: 4. In one embodiment the variant of the fragment of human ACE2 comprises a L221C mutation and/or a H222L mutation, the numbering referring to SEQ ID NO: 4. It has been shown that a cysteine at position 221 is important for the formation of polymeric antibodies. The histidine at position 222 may be mutated to leucine, since a histidine at this position may interfere with disulfide bond formation and thereby with polymeric assembly (Mekhaiel et al. (2011) Scientific Reports 1, Article Number 124).

“A fragment of the Fc portion of human IgM” refers to a polypeptide which lacks one or more amino acids compared to the full-length sequence of the Fc region of human IgM according to SEQ ID NO: 4. The fragment of the Fc region of human IgM is capable of forming multimers, preferably hexamers. The formation of multimers can be determined by size exclusion chromatography coupled to multi-angle light scattering (SEC-MALS) as described in the examples herein.

In one embodiment, the fragment of the Fc portion of human IgM consists of SEQ ID NO: 17. In one embodiment, the fragment of the Fc portion of human IgM consists of SEQ ID NO: 18. In one embodiment, the fragment of the Fc portion of human IgM consists of SEQ ID NO: 19. In one embodiment, the fragment of the Fc portion of human IgM consists of SEQ ID NO: 20 In one embodiment, fragment of the Fc portion of human IgM consists of the sequence according to SEQ ID NO: 7. In one embodiment, the fragment of the Fc portion of human IgM consists of SEQ ID NO: 21. Within the present application, the term “cm4” is used as a synonym for the term “cμ4” and both cm4 and cμ4 refer to the sequence according to SEQ ID NO: 7.

In one embodiment, the second part of the fusion protein of the present invention comprises the Fc portion of human IgG2 or a variant thereof. The Fc portion of human IgG2 comprises the CH2 and CH3 domains of human IgG2 linked together to form the Fc portion. In a full-length human IgG2 antibody the Fc portion is connected to the Fab fragment through a hinge region. The Fab fragment comprises the heavy chain variable region and the CH1 domain. Preferably, the Fc portion of human IgG2 used in the fusion protein of the present invention has the sequence according to SEQ ID NO: 10.

The Fc portion of human IgG2 has lower ability to bind to Fc receptors and recruit the immune system (Stewart et al. (2014) Journal of Immunotherapy of Cancer 1-10, available at https://doi.org/10.1186/s40425-014-0029-x). Hence, using the Fc portion of human IgG2 instead of the Fc portion of human IgG1 could be beneficial in the development of a medicament.

“A variant of the Fc portion of human IgG2” refers to the Fc portion of human IgG2 which has one or more amino acid substitutions compared to the wild-type Fc portion of human IgG2 according to SEQ ID NO: 10. In one embodiment, the one or more amino acid substitutions lead to decreased effector functions compared to the wild-type Fc portion of human IgG2 according to SEQ ID NO: 10. In one embodiment, the variant of the Fc portion of human IgG2 has one to twelve, one to eleven, one to ten, one to nine, one to eight, one to seven, one to six, one to five, one to four, one to three, one or two amino acid substitutions compared to the wild-type Fc portion of human IgG2 according to SEQ ID NO: 10. In one embodiment, the variant of the Fc portion of human IgG2 has one, two, three, four, five, six, seven, eight, nine, ten, eleven or twelve amino acid substitutions compared to the wild-type Fc portion of human IgG2 according to SEQ ID NO: 10.

In one embodiment, the variant of the Fc portion of human IgG2 comprises a mutation at at least one position selected from the positions 16, 18, 19, 49, 90, 111 and 112, the numbering referring to SEQ ID NO: 10. In one embodiment, the variant of the Fc portion of human IgG2 comprises a mutation at positions 16, 18, 19, 49, 90, 111 and 112, the numbering referring to SEQ ID NO: 10. In one embodiment, the variant of the Fc portion of human IgG2 comprises at least one mutation selected from the group consisting of V16A, G18A, P19S, H49A, V90L, A111S and P112S. In one embodiment, the variant of the Fc portion of human IgG2 comprises the mutations V16A, G18A, P19S, H49A, V90L, A111 S and P112S. It has been shown that these mutations eliminate all effector functions of human IgG2 (Vafa et al. (2014) Methods 65: 114-126. In one embodiment, the variant of the Fc portion of human IgG2 comprises the tailpiece of human IgM or human IgA. Such variants are described in Sorensen et al. (1996) J. Immunol. 156(8): 2858-2865.

In one embodiment, the first and the second part of the fusion protein of the present invention are linked by a linker sequence. The linker sequence is a short amino acid sequence which does not have a function on its own and which does not affect the folding of the fusion protein. In one embodiment, the linker sequence comprises five to twenty amino acids, preferably six to 18 amino acids, more preferably seven to 17 amino acids and most preferably eight amino acids.

In one embodiment, the linker sequence consists of small amino acids selected from glycine and serine. An overview of linker sequences is provided in Chen et al. (2013) Adv. Drug Deliv. Rev. 65(10): 1357-1369. In one embodiment, the linker sequence comprises five to twenty amino acids and consists of small amino acids selected from glycine and serine. In one embodiment, the linker sequence comprises six to 18 amino acids and consists of small amino acids selected from glycine and serine. In one embodiment, the linker sequence comprises seven to 17 amino acids and consists of small amino acids selected from glycine and serine. In one embodiment, the linker sequence comprises eight amino acids and consists of small amino acids selected from glycine and serine. In one embodiment, the linker sequence has the amino acid sequence according to SEQ ID NO: 5.

In another embodiment, the linker sequence is the hinge region of a human IgG antibody. In one embodiment, the linker sequence is the hinge region of a human IgG1 antibody, a human IgG4 antibody or a human IgG2 antibody. In one embodiment, the hinge region is the hinge region of a human IgG4 antibody and has the amino acid sequence according to SEQ ID NO: 14. In one embodiment, the hinge region is the hinge region of a human IgG1 antibody and has the amino acid sequence according to SEQ ID NO: 15. In one embodiment, the hinge region is the hinge region of a human IgG2 antibody and has the amino acid sequence according to SEQ ID NO: 16. An overview of antibody structure and the role of the hinge region is provided in the Chapter “Antibody structure-function relationships” in Therapeutic Antibody Engineering-Current and Future Advances Driving the Strongest Growth Area in the Pharmaceutical Industry, Woodhead Publishing Series in Biomedicine 2012, pages 37-56, 459-595 (available at Antibody structure-function relationships-ScienceDirect).

In a fusion protein with the Fc part of IgG2 or a variant or fragment thereof preferably the hinge region of a human IgG2 antibody is used. More preferably, the hinge region of a human IgG2 antibody according to SEQ ID No: 16 is used in a fusion protein with the Fc part of IgG2 or a variant or fragment thereof.

In one embodiment, the fusion protein of the present invention comprises a signal peptide which is located N-terminal of the ACE2 part of the fusion protein. The signal peptide functions to target the protein to the endoplasmic reticulum and ultimately to secretion from the cell. The skilled person knows suitable signal peptides. In one embodiment, the signal peptide is selected from a human albumin signal peptide, a human chymotrypsinogen signal peptide, a human interleukin-2 signal peptide, a human trypsinogen-2 signal peptide or a heavy or light chain signal peptide. In a preferred embodiment, the signal peptide is a human albumin signal peptide. In a particularly preferred embodiment, the signal peptide is a human albumin signal peptide according to SEQ ID NO: 6.

In a particular embodiment, the fusion protein of the present invention has the amino acid sequence according to SEQ ID NO: 8 which comprises the signal peptide according to SEQ ID NO: 6, amino acids 18 to 740 of human ACE2 (SEQ ID NO: 3), the linker sequence according to SEQ ID NO: 5 and the Fc portion of human IgM according to SEQ ID NO: 4.

In a particular embodiment, the fusion protein of the present invention comprises amino acids 18 to 740 of human ACE2 (SEQ ID NO: 3), the linker sequence according to SEQ ID NO: 5 and the Fc portion of human IgM according to SEQ ID NO: 4.

In a particular embodiment, the fusion protein of the present invention comprises amino acids 18 to 740 of human ACE2 (SEQ ID NO: 3), a linker sequence and the Fc portion of human IgM according to SEQ ID NO: 4.

In another particular embodiment, the fusion protein of the present invention has the amino acid sequence according to SEQ ID NO: 9 which comprises the signal peptide according to SEQ ID NO: 6, amino acids 18 to 740 of human ACE2 (SEQ ID NO: 3), the linker sequence according to SEQ ID NO: 5 and the fragment of the Fc portion of human IgM according to SEQ ID NO: 7.

In another particular embodiment, the fusion protein of the present invention comprises amino acids 18 to 740 of human ACE2 (SEQ ID NO: 3), the linker sequence according to SEQ ID NO: 5 and the fragment of the Fc portion of human IgM according to SEQ ID NO: 7.

In another particular embodiment, the fusion protein of the present invention comprises amino acids 18 to 740 of human ACE2 (SEQ ID NO: 3), a linker sequence and the fragment of the Fc portion of human IgM according to SEQ ID NO: 7.

In a particular embodiment, the fusion protein of the present invention has the amino acid sequence according to SEQ ID NO: 11 which comprises the signal peptide according to SEQ ID NO: 6, amino acids 18 to 740 of human ACE2 (SEQ ID NO: 3), the linker sequence according to SEQ ID NO: 5 and the Fc portion of human IgG2 according to SEQ ID NO: 10.

In a particular embodiment, the fusion protein of the present invention comprises amino acids 18 to 740 of human ACE2 (SEQ ID NO: 3), the linker sequence according to SEQ ID NO: 5 and the Fc portion of human IgG2 according to SEQ ID NO: 10.

In a particular embodiment, the fusion protein of the present invention comprises amino acids 18 to 740 of human ACE2 (SEQ ID NO: 3), a linker sequence and the Fc portion of human IgG2 according to SEQ ID NO: 10.

The present invention also relates to multimers of the fusion protein of a fragment of human ACE2 and the Fc portion of human IgM or a fragment of the Fc portion of human IgM. The term “multimer” is intended to mean a structure wherein two to twelve fusion proteins of the present invention are associated by disulfide bonds. In one particular embodiment, the term multimer means hexamer, i.e. six fusion proteins of the present invention are associated by disulfide bonds. It was surprisingly shown that the IgM fusion proteins of the present invention are able to predominantly form hexamers. By the formation of hexamers the antigen binding can be enhanced.

In one embodiment, the present invention also relates to a composition comprising predominantly the hexameric form of the fusion protein of a fragment of human ACE2 and the Fc portion of human IgM or a fragment of the Fc portion of human IgM, i.e. at least 51% or 55%, preferably at least 60% or 65%, more preferably at least 70% or 75%% and most preferably 80%, 85% or 90% of the fusion protein of a fragment of human ACE2 and the Fc portion of human IgM or a fragment of the Fc portion of human IgM in the composition is present as a hexamer. Methods for separating the hexamer made of six homodimers from the free homodimers and for distinguishing between homodimers and hexamer of homodimers are described herein and include affinity chromatography and size exclusion chromatography.

The present invention also relates to a nucleic acid molecule comprising a nucleic acid sequence encoding the fusion protein of the present invention. The skilled person knows how to construct a nucleic acid molecule when the amino acid sequence of a protein is known. In particular, the construction of the nucleic acid molecule involves back-translating the amino acid sequence of the protein into a nucleic acid sequence using the three-letter genetic code and optionally taking into account the codon usage of the host cell in which the protein is to be expressed using the nucleic acid molecule.

In one embodiment, the nucleic acid molecule comprising a nucleic acid sequence encoding the fusion protein of the present invention is an isolated nucleic acid molecule. The term “isolated nucleic acid molecule” refers to a nucleic acid molecule that is identified and separated from at least one contaminant nucleic acid molecule with which it is ordinarily associated in the environment in which it was produced. Preferably, the isolated nucleic acid is free of association with all components associated with the production environment.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

Expression vectors contain elements for the expression of the nucleic acids such as suitable promoters and polyadenylation signals. In addition, the expression vectors typically contain a selection marker gene under the control of suitable promoter to enable the distinction of cells which contain the expression vector from cells which do not contain the expression vector. The elements and methods needed to construct expression vectors which are suitable for expressing a recombinant protein such as the fusion protein of the present invention are well-known to the skilled person and described for example in Makrides et al. (1999) Protein Expr. Purif. 17: 183-202 and Kaufman (2000) Mol. Biotechnol. 16: 151-161.

The expression vector is used to transform, i.e. genetically modify, suitable host cells. The skilled person is aware of methods for introducing the expression vectors into the mammalian cells. These methods include the use of commercially available transfection kits such as Lipofectamine® of ThermoFisher, PEImax of Polyplus Sciences), 293-Free transfection reagent (Millipore) or Freestyle Max of Invitrogen. Further suitable methods include electroporation, calcium phosphate-mediated transfection and DEAE-dextrane transfection. After transfection the cells are subjected to selection by treatment with a suitable agent based on the selection marker(s) encoded by the expression vector(s) to identify the stably transfected cells which contain the recombinant nucleic acid molecule.

The terms “host cell,” “host cell line,” and “host cell culture” are used interchangeably and refer to cells into which an exogenous nucleic acid or an expression vector has been introduced, including the progeny of such cells. Host cells include “transformants” and “transformed cells,” which include the primary transformed cell and progeny derived therefrom without regard to the number of passages.

The fusion protein of the present invention is preferably produced in mammalian host cells. Suitable mammalian host cells for expressing the fusion protein of the invention include Chinese Hamster Ovary (CHO) cells (including dhfr negative CHO cells used with a DHFR selectable marker), NS0 myeloma cells, COS cells, SP2 cells, monkey kidney CV1, human embryonic kidney line 293, baby hamster kidney cells (BHK), mouse Sertoli cells (TM4), African green monkey kidney cells (VERO-76), human cervical carcinoma cells (HELA), canine kidney cells (MDC), buffalo rat liver cells (BRL 3 A), human lung cells (W138), human liver cells (Hep G2), mouse mammary tumor cells (MMT 060562), TRI cells, MRC 5 cells and FS4 cells. More preferably, the host cells are derived from a rodent. Most preferably, the mammalian cells are Chinese hamster ovary (CHO) or 293 cells. In one embodiment the CHO cells are ExpiCHO cells. In one embodiment the 293 cells are Expi293 cells.

To produce the fusion protein of the present invention, the host cells are cultured in a suitable culture medium.

The terms “medium”, “cell culture medium” and “culture medium” are interchangeably used herein and refer to a solution containing nutrients which are required for growing mammalian cells. Typically, a cell culture medium provides essential and non-essential amino acids, vitamins, energy sources, lipids, and trace elements required by the cell for minimal growth and/or survival. The cell culture medium may also comprise growth factors. Preferably, the medium is chemically defined in that all its components and their concentration are known. Also preferably, the medium is serum-free and hydrolysate-free and does not contain any components derived from animals. In a more preferred embodiment the medium is serum-free and hydrolysate-free and does not contain any components derived from animals or insulin.

In one embodiment the medium used in the method of the present invention is a commercially available medium such as FreeStyle 293 expression medium (Life Technologies), PoICHO P Powder Base CD, ActiPro (both available from GE), PowerCHO-2, ProCHO-5 (both available from Lonza) or EX-CELL® Advanced CHO fed batch medium (available from Sigma). If the cells used for expressing the fusion protein are ExpiCHO cells, the medium is ExpiCHO medium available from ThermoFisher. If the cells used for expressing the fusion protein are Expi293 cells, the medium is Expi293 medium available from ThermoFisher.

For culturing the mammalian cells different strategies are available, including batch culture, perfusion culture, continuous culture and fed-batch culture. Preferably, a fed-batch culture process is used. In fed-batch culture the culturing process is started with a certain volume of the basal medium and one or more feed media comprising one or more nutrients are fed at later time-point(s) of the culture process to prevent nutrient depletion while no product is removed from the cell culture broth. Accordingly, the term “feeding” means that at least one component is added to an existing culture of cells.

The term “basal medium” is intended to refer to the medium which is used from the beginning of the cell culture process. The mammalian cells are inoculated into the basal medium and grown in this medium for a certain period until the feeding is started. The basal medium meets the definition of the culture medium as provided above. If a commercially available medium is used, additional components may be added to the basal medium.

The feed medium is added to the cell culture after the cells have been cultured in the basal medium for a certain period. The feed medium serves to prevent nutrient depletion and therefore may not have the same composition as the basal medium. In particular, the concentration of one or more nutrients may be higher in the feed medium than in the basal medium. In one embodiment, the feed medium has the same composition as the basal medium. In another embodiment, the feed medium has another composition as the basal medium. The feed medium may be added continuously or as a bolus at defined time points.

Suitable feed media are known to the skilled person and include PoICHO Feed-A Powder Base CD, PoICHO Feed-B Powder Base CD, Cell Boost 7a and Cell Boost 7b (all available from GE), BalanCD® CHO Feed 3 Medium (available from Irvine Scientific) and EX-CELL® Advanced CHO feed 1 (available from Sigma).

The culturing of the host cell may be performed at a constant temperature, e.g. at a temperature of 37° C.±0.2° C. Alternatively, the culture temperature may be reduced from a first temperature to a second temperature, i.e. the temperature is actively downregulated. Hence, the second temperature is lower than the first temperature. The first temperature may be 37° C.±0.2° C. The second temperature may be in the range of from 30° C. to 36° C.

After the fusion protein of the present invention has been produced by culturing the host cell in a suitable culture medium, the fusion protein is harvested from the cell culture. Since Fc fusion proteins expressed from mammalian cells are typically secreted into the cell culture fluid during the cultivation process, the product harvest at the end of the cultivation process occurs by separating cell culture fluid comprising the fusion protein from the cells. The cell separation method should be gentle to minimize cell disruption to avoid the increase of cell debris and release of proteases and other molecules that could affect the quality of the fusion protein product. Usually, the harvesting of the cell culture fluid comprising the fusion protein involves centrifugation and/or filtration, whereby the fusion protein is present in the supernatant and the filtrate, respectively. Expanded bed adsorption chromatography is an alternative method to avoid centrifugation/filtration methods.

After harvesting the cell culture fluid comprising the fusion protein the fusion protein has to be purified from the cell culture fluid. The purification of Fc fusion proteins is usually accomplished by a series of standard techniques that can include chromatographic steps such as anion exchange chromatography, cation exchange chromatography, affinity chromatography, hydrophobic interaction chromatography, hydroxyapatite chromatography and size exclusion chromatography. The affinity chromatography may comprise protein A affinity chromatography for IgG2 fusion proteins and affinity chromatography with IgM affinity ligands such as CaptureSelect IgM affinity resin (ThermoFisher) for IgM fusion proteins and fusion proteins comprising at least the cm4 part of IgM. Further, the purification process may comprise one or more ultra-, nano- or diafiltration as well as tangential flow filtration and/or cross flow filtration steps.

After purifying the fusion protein it can be used to prepare a pharmaceutical composition. A pharmaceutical composition is a composition which is intended to be delivered to a patient for treating or preventing a disease or condition. In addition to the active agent, i.e. the fusion protein of the present invention, a pharmaceutical composition typically contains at least one pharmaceutically acceptable excipient. Pharmaceutically acceptable excipients are substances which do not interfere with the physiological activity of the fusion protein and which stabilize the pharmaceutical composition and/or enhance solubility or decrease viscosity of the pharmaceutical composition. Typical pharmaceutically acceptable excipients for recombinant proteins include buffers, salts, sugars or sugar alcohols, amino acids and surface-active agents.

The pharmaceutical composition comprises a therapeutically effective amount of the fusion protein or the multimer of the present invention. The term “therapeutically effective amount” refers to an amount of the fusion protein or the multimer of the present invention sufficient to treat a specified disorder, condition or disease such as ameliorate, palliate, lessen, and/or delay one or more of its symptoms. With respect to an infection with a coronavirus and in particular SARS-CoV-2 the therapeutically effective amount of the fusion protein or the multimer of the present invention ameliorates, palliates, lessens, and/or delays one or more of symptoms selected from coughing, shortness of breath, difficulty breathing, fever, chills, tiredness, muscle aches, sore throat, headache, chest pain and loss of smell and/or taste. A therapeutically effective amount can be administered in one or more administrations.

The fusion protein or the multimer of the present invention is for medical use, i.e. it is intended to be used to prevent and/or treat a disease.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, decreasing the dose of one or more other medications required to treat the disease, and/or prolonging survival. The use of the present invention contemplates any one or more of these aspects of treatment.

The term “prevent,” and similar words such as “prevented”, “preventing” etc., indicate an approach for preventing, inhibiting, or reducing the likelihood of the recurrence of, a disease or condition. It also refers to delaying the recurrence of a disease or condition or delaying the recurrence of the symptoms of a disease or condition. As used herein, “prevention” and similar terms also includes reducing the intensity, effect, symptoms and/or burden of a disease or condition prior to recurrence of the disease or condition.

In one embodiment, the fusion protein or the multimer of the present invention is used to prevent and/or treat an infection with a coronavirus binding to ACE2. Coronaviruses are enveloped viruses with a positive sense, single-stranded RNA genome and an icosahedral protein shell. The Spike protein consisting of the S1 and S2 subunits forms a homotrimer which projects from the envelope and mediates the interaction with the target cells by binding to ACE2. Coronaviruses often cause respiratory diseases in humans and other mammalian as well as bird species. In humans, seven coronavirus strains are known: HCoV-OC43, HCoV-HKU1, HCoV-229E, HCoV-NL63, MERS-CoV, SARS-CoV and SARS-CoV-2. The first four coronavirus strains (HCoV-OC43, HCoV-HKU1, HCoV-229E, HCoV-NL63) cause only mild symptoms, whereas infection with MERS-CoV, SARS-CoV and SARS-CoV-2 may lead to severe, potentially life-threatening disease.

It has been shown that SARS-CoV, SARS-CoV-2 and HCoV-NL63 bind to ACE2 and use this binding to enter the target cells (Li et al. (2003) Nature 426(6965): 450-4; Hoffmann et al. (2020) Cell 181: 1-10; Hofmann et al. (2005) Proc Natl Acad Sci USA. 102(22):7988-93). Accordingly, the fusion protein or the multimer of the present invention can be used to treat and/or prevent infection with a coronavirus binding to ACE2, in particular infection with SARS-CoV, SARS-CoV-2 or HCoV-NL63. Further coronaviruses binding to ACE2 can be identified by inoculating cells expressing ACE2 either transiently or constitutively with pseudotyped VSV (vesicular stomatitis virus) expressing the coronavirus Spike protein and a reporter protein and detecting the activity of the reporter protein after the inoculation period (see protocol in Hoffmann et al. (2020) Cell 181: 1-10). In one embodiment, the fusion protein or the multimer of the present invention is used to treat and/or prevent infection with a coronavirus binding to ACE2, wherein the coronavirus binding to ACE2 is not SARS-CoV. In one embodiment, the fusion protein or the multimer of the present invention is used to treat and/or prevent infection with a coronavirus binding to ACE2, wherein the coronavirus binding to ACE2 is SARS-CoV.

In one embodiment, the fusion protein or the multimer of the present invention is used to treat and/or prevent infection with a coronavirus binding to ACE2, wherein the coronavirus binding to ACE2 is SARS-CoV-2 or a variant of SARS-CoV-2 comprising the amino acid substitution D614G and/or the amino acid substitution N439K. The variant of SARS-CoV-2 comprising the amino acid substitution D614G is described in Korber et al. (2020) Cell 182(4): 812-827 and the amino acid substitution N439K is described in Thomson et al. (2021) Cell 184(5): 1171, 1187.e20; available at https://doi.org/10.1101/2020.11.04.355842. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitution D614G. The amino acid substitution D614G is caused by an A-to-G nucleotide mutation at position 23,403 in the Wuhan reference strain. The numbering of the amino acids in the variants refers to the numbering in the Spike protein of SARS-CoV-2 according to SEQ ID NO: 13. Hence, a SARS-CoV-2 virus with the Spike protein according to SEQ ID NO: 13 is defined to be the wild-type SARS-CoV-2 from which any variants are derived.

In one embodiment, the fusion protein or the multimer of the present invention is used to treat and/or prevent infection with a coronavirus binding to ACE2, wherein the coronavirus binding to ACE2 is a variant of SARS-CoV-2 comprising at least one amino acid substitution selected from the group consisting of L452R, E484K, K417N and N501Y. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions E484K, K417N and N501Y.

In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitution D614G and at least one additional amino acid substitution. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions D614G, N501Y, A570D, P681H, T7161, S982A and D1118H and comprising a deletion of amino acids 69, 70 and 145. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions D614G, Y453F, 1692V and M12291 and comprising a deletion of amino acids 69 and 70. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions D614G, S131, W152C and L452R. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions D614G, E484K and V1176F. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions D614G, L18F, T20N, P26S, D138Y, R190S, K417T, E484K, N501Y, H655Y, T10271 and V1176F. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions D614G, D80A, D215G, K417N, E484K, N501Y and A701V and comprising a deletion of amino acids 242, 243 and 244. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions D614G, L18F, D80A, D215G, K417N, E484K, N501Y and A701V and comprising a deletion of amino acids 242, 243 and 244. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions D614G, D80A, R246I, K417N, E484K, N501Y and A701V and comprising a deletion of amino acids 242, 243 and 244. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions E484Q and L452R. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions E484K and D614G and comprising a deletion of amino acids 145 and 146. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions D614G, T478K, P681R and L452R and not comprising amino acid substitution E484Q. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions T19R, D614G, T478K, P681R, D950N and L452R and comprising a deletion of amino acids 157 and 158 and not comprising amino acid substitution E484Q. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions G75V, T76I, L452Q, F490S, D614G and T859N and comprising a deletion of amino acids 246 to 252. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitutions K417N, S477N, E484A, N501Y, D614G, H655Y and P681H (B.1.1.529; Omicron) and optionally further comprising the amino acid substitutions L452R and F486V.

The numbering of the amino acids in the variants refers to the numbering in the Spike protein of SARS-CoV-2 according to SEQ ID NO: 13. For the purposes of defining variants, the amino acid sequence according to SEQ ID NO: 13 is considered to be the wild-type sequence of the Spike protein of SARS-CoV-2.

In one embodiment, the fusion protein or the multimer of the present invention is used to treat and/or prevent infection with a coronavirus binding to ACE2, wherein the coronavirus binding to ACE2 is a variant of SARS-CoV-2 comprising one or more amino acid substitutions in the receptor-binding domain of the Spike protein of SARS-CoV-2. The receptor-binding domain of the Spike protein of SARS-CoV-2 comprises amino acids 331 to 524 of SEQ ID NO: 13 (see Tai et al. (2020) Cell. Mol. Immunol. 17: 613-620). In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitution N501Y. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitution E484K. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitution K417T/N. In one embodiment, the fusion protein or the multimer of the present invention is used to treat and/or prevent infection with a coronavirus binding to ACE2, wherein the coronavirus binding to ACE2 is a variant of SARS-CoV-2 comprising the amino acid substitution L452R.

In one embodiment, the fusion protein or the multimer of the present invention is used to treat and/or prevent infection with a coronavirus binding to ACE2, wherein the coronavirus binding to ACE2 is a variant of SARS-CoV-2 which has a higher binding affinity to ACE2 compared to the SARS-CoV-2 comprising the wild-type Spike protein according to SEQ ID NO: 13. The affinity of a variant of SARS-CoV-2 to ACE2 can for example be determined using a pseudovirus assay. The pseudovirus assay uses a lentivirus which is pseudotyped with the S protein of wild-type SARS-CoV-2 or of a variant thereof and which contains a reporter gene such as the luciferase gene. Such lentiviruses can be obtained for example from BPS Bioscience. The pseudotyped lentivirus is incubated with ACE2 expressing cells to allow the virus to enter the cells and to express the reporter gene. If the expression of the reporter gene from a lentivirus pseudotyped with the S protein of a variant SARS-CoV-2 is higher than the expression of the reporter gene from a lentivirus pseudotyped with the S protein of wild-type SARS-CoV-2, the variant has a higher binding affinity to ACE2. It is assumed that the fusion proteins of the present invention have a higher affinity to those variants which have a higher binding affinity to ACE2, such as the variant B.1.1.7.

In one embodiment, the fusion protein or the multimer of the present invention is used to treat and/or prevent infection with a coronavirus binding to ACE2, wherein the coronavirus binding to ACE2 is a variant of SARS-CoV-2 which has a higher transmissibility compared to the SARS-CoV-2 comprising the wild-type Spike protein according to SEQ ID NO: 13. The viral transmissibility can be determined using the basic reproduction number R₀ which is the average number of people who will catch a disease from one contagious person.

The route of administration is in accordance with known and accepted methods, e.g., injection or infusion by subcutaneous, intravenous, intraperitoneal, intramuscular, intra-arterial, intralesional or intraarticular routes. In another embodiment the fusion protein or the multimer of the present invention is to be administered intranasally, e.g. by means of a nasal spray, a nasal ointment or nasal drops. In another embodiment, the fusion protein or the multimer of the present invention is administered by topical administration or by inhalation. Preferably, the fusion protein or the multimer of the present invention is administered by intravenous injection or infusion.

Dosages and desired drug concentrations of pharmaceutical compositions of the present invention may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

In one embodiment, the fusion protein or the multimer of the present invention is administered intravenously at a dosage of 0.1 mg/kg body weight to 4 mg/kg body weight, such as a dosage of 0.1 mg/kg body weight, 0.2 mg/kg body weight, 0.3 mg/kg body weight, 0.4 mg/kg body weight, 0.5 mg/kg body weight, 0.6 mg/kg body weight, 0.7 mg/kg body weight, 0.8 mg/kg body weight, 0.9 mg/kg body weight, 1.0 mg/kg body weight, 1.1 mg/kg body weight, 1.2 mg/kg body weight, 1.3 mg/kg body weight, 1.4 mg/kg body weight, 1.5 mg/kg body weight, 1.6 mg/kg body weight, 1.7 mg/kg body weight, 1.8 mg/kg body weight, 1.9 mg/kg body weight, 2.0 mg/kg body weight, 2.1 mg/kg body weight, 2.2 mg/kg body weight, 2.3 mg/kg body weight, 2.4 mg/kg body weight, 2.5 mg/kg body weight, 2.6 mg/kg body weight, 2.7 mg/kg body weight, 2.8 mg/kg body weight, 2.9 mg/kg body weight, 3.0 mg/kg body weight, 3.1 mg/kg body weight, 3.2 mg/kg body weight, 3.3 mg/kg body weight, 3.4 mg/kg body weight, 3.5 mg/kg body weight, 3.6 mg/kg body weight, 3.7 mg/kg body weight, 3.8 mg/kg body weight, 3.9 mg/kg body weight or 4.0 mg/kg body weight.

In one embodiment, the fusion protein or the multimer of the present invention is administered intravenously at a dosage of 10 mg/kg body weight to 150 mg/kg body weight, such as a dosage of 10 mg/kg body weight, 15 mg/kg body weight, 20 mg/kg body weight, 25 mg/kg body weight, 30 mg/kg body weight, 35 mg/kg body weight, 40 mg/kg body weight, 45 mg/kg body weight, 50 mg/kg body weight, 55 mg/kg body weight, 60 mg/kg body weight, 65 mg/kg body weight, 70 mg/kg body weight, 75 mg/kg body weight, 80 mg/kg body weight, 85 mg/kg body weight, 90 mg/kg body weight, 95 mg/kg body weight, 100 mg/kg body weight, 105 mg/kg body weight, 110 mg/kg body weight, 115 mg/kg body weight, 120 mg/kg body weight, 125 mg/kg body weight, 130 mg/kg body weight, 135 mg/kg body weight, 140 mg/kg body weight, 145 mg/kg body weight or 150 mg/kg body weight.

The fusion protein or multimer may be administered once per day, twice per day, three times per day, every other day, once per week or once every two weeks.

The fusion protein or multimer may be administered for a period of three days, four days, five days, six days, seven days, eight days, nine days or ten days.

By administering the fusion protein or multimer of the present invention, the infection with a coronavirus and in particular with SARS-CoV-2 is treated, i.e. at least one of the symptoms of an infection with SARS-CoV-2 is reduced or abolished. Symptoms of an infection with SARS-CoV-2 include coughing, shortness of breath, difficulty breathing, fever, chills, tiredness, muscle aches, sore throat, headache, chest pain and loss of smell and/or taste. In one embodiment, by the administration of the fusion protein or multimer of the present invention the fever caused by infection with SARS-CoV-2 is reduced. In one embodiment, the administration of the fusion protein or multimer of the present invention to a subject reduces the risk that the subject experiences a severe course of infection with SARS-CoV-2. In one embodiment, the administration of the fusion protein or multimer of the present invention to a subject reduces the risk that the subject experiences multi-organ failure, acute respiratory distress syndrome (ARDS) or pneumonia. In one embodiment, the administration of the fusion protein or multimer of the present invention to a subject reduces the risk that the subject experiences long-term effects of the infection with SARS-CoV-2 such as lung damage, neurological disorders, dermatological disorders and cardiovascular disease. In one embodiment, the administration of the fusion protein or multimer of the present invention to a subject reduces the concentration of the cytokines IL6 and/or IL8 in the blood. In one embodiment, the administration of the fusion protein or multimer of the present invention to a subject reduces the concentration of SARS-CoV-2 virus particles in the blood. In one embodiment, the administration of the fusion protein or multimer of the present invention to a subject stimulates the production of antiviral antibodies. In one embodiment, the administration of the fusion protein or multimer of the present invention to a subject stimulates the production of antiviral IgA and/or IgG antibodies.

In one embodiment, the fusion protein or multimer of the present invention is administered to a subject suffering from a severe infection with SARS-CoV-2. In one embodiment, the fusion protein or multimer of the present invention is administered to a subject infected with SARS-CoV-2 and requiring artificial ventilation. In one embodiment, the fusion protein or multimer of the present invention is administered to a subject infected with SARS-CoV-2 and requiring extracorporeal membrane oxygenation (ECMO).

By administering the fusion protein or multimer of the present invention, the infection with a coronavirus and in particular with SARS-CoV-2 is prevented, i.e. the treated subject does not develop symptoms of an infection with SARS-CoV-2.

In one embodiment, the fusion protein or multimer of the present invention is administered to a subject which has been in contact with a subject infected with SARS-CoV-2, but has not yet developed any symptoms of SARS-CoV2 infection. Subjects which have been in contact with a subject infected with SARS-CoV-2 can be identified by use of a “Corona warning app” installed on the smartphone.

In one embodiment, the fusion protein or multimer of the present invention is administered to a subject for which a test with a throat or nasal swab of said subject indicates that it is infected with SARS-CoV-2, but which has not developed any symptoms of an infection with SARS-CoV-2.

In the treatment or prevention of an infection with a coronavirus binding to ACE2 and in particular SARS-CoV-2 the fusion protein or multimer of the present invention may be combined with a known anti-viral agent. Anti-viral agents are medicaments used to treat viral infections and include both specific anti-viral agents and broad-spectrum viral agents. Suitable anti-viral agents include, but are not limited to, nucleoside analogs, inhibitors of viral protease, inhibitors of viral polymerase, blockers of virus entry into the cell, Janus kinase inhibitors, but also inhibitors of inflammatory mediators.

In specific embodiments, the anti-viral agent is selected from the group consisting of remdesivir, arbidol HCl, ritonavir, lopinavir, darunavir, ribavirin, chloroquin and derivatives thereof such as hydroxychloroquin, nitazoxanide, camostat mesilate, anti-IL6 and anti-IL6 receptor antibodies such as tocilizumab, siltuximab and sarilumab and baricitinib phosphate.

In the treatment or prevention of an infection with SARS-CoV-2 the fusion protein or multimer of the present invention may be combined with an anti-SARS-CoV-2 monoclonal antibody. Anti-SARS-CoV2 monoclonal antibodies include, but are not limited to, LY-CoV555 (LY3819253) developed by Eli Lilly and Company, REGN-COV2 which is a cocktail of REGN10933 and REGN10987 and which is developed by Regeneron, VIR-7831 (GSK4182136) developed by Vir Biotechnology and GlaxoSmithKline, CT-P59 developed by Celltrion, AZD 7442 which is a combination of antibodies AZD8895 and AZD1061 and which is developed by Astra Zeneca, JS016 developed by Junshi Biosciences, TY027 developed by Tychan Pte Ltd, BRII-96 and BRII-98 developed by Brii Biosciences, SCTA01 developed by Sinocelltech Ltd, ADM03820 developed by Ology Bioservices, BI767551 developed by Boehringer Ingelheim and others and COR-101 developed by Corat Therapeutics.

Apart from its function in binding coronaviruses, ACE2 has also been implicated in several disorders and diseases such as hypertension (including high blood pressure), congestive heart failure, chronic heart failure, acute heart failure, contractile heart failure, myocardial infarction, arteriosclerosis, kidney failure, renal failure, Acute Respiratory Distress Syndrome (ARDS), Acute Lung Injury (ALI), chronic obstructive pulmonary disease (COPD), pulmonary hypertension, renal fibrosis, chronic renal failure, acute renal failure, acute kidney injury, inflammatory bowel disease and multi-organ dysfunction syndrome. Hence, the fusion protein or multimer of the present invention can also be used in the treatment of these disorders and diseases.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

The detailed description is merely exemplary in nature and is not intended to limit application and uses. The following examples further illustrate the present invention without, however, limiting the scope of the invention thereto. Various changes and modifications can be made by those skilled in the art on the basis of the description of the invention, and such changes and modifications are also included in the present invention.

### EXAMPLES

### A. Materials and Methods

### 1. Production and Purification of the ACE2-Fc Fusion Proteins

The genes coding the sequences (ACE2-Fc (IgM) (SEQ ID NO: 8), ACE2-Fc (IgG2) (SEQ ID NO: 11) and ACE2-Fc (cm4) (SEQ ID NO: 9)) were ordered and obtained from a commercial gene synthesis provider. The codons were optimized for Homo sapiens with the Gene optimization tool from GeneArt. Kozak sequence was added before the start codon. A stop codon was added after the codon that encodes the last amino acid in the sequence. The synthesized genes were delivered in a pcDNA3.1(+) plasmid. The genes have HindIII and Xhol restriction sites. Larger amounts of the delivered plasmids were produced and isolated by standard techniques with XL-1 bacterial cells. The purified plasmids were sent for sequencing with several primers. The entire range of the gene encoding the ACE2-Fc fusion protein was verified.

The ACE2-Fc fusion proteins were produced by transient expression in Expi293 or ExpiCHO cells after transfection with Expifectamine using the manufacturer's protocol. After 6 days, the supernatant was collected, and the ACE2-Fc fusion proteins were purified by a combination of two standard techniques—affinity chromatography followed by size-exclusion chromatography. For the ACE2-Fc (IgG2) fusion protein a protein A chromatography step was applied as affinity chromatography. For the ACE2-Fc (IgM) fusion protein a chromatography step using CaptureSelect IgM resin (ThermoFisher) was applied as affinity chromatography. The unbound sample was washed off with PBS. The bound proteins were eluted with cold 0.1 M glycine buffer pH 3.0 into ⅕ volume of cold 1 M Tris buffer pH 8.5. After affinity chromatography, the ACE2 fusion proteins were further purified and buffer-exchanged by size-exclusion chromatography on a HiPrep™ 16/60 Sephacryl® S-500 HR column (Cytiva) using PBS as the eluent. The amounts of ACE2-Fc fusion proteins that are obtained from transient transfection of 1 L cell culture with the exemplary sequence are in the range of tens to hundreds of milligrams.

The secondary and tertiary structure of the ACE2-FC fusion proteins was studied by circular dichroism (CD). It was shown that the fusion proteins are folded (data not shown).

The quaternary structure and oligomeric state were studied with size-exclusion chromatography coupled to multi-angle light scattering. The system consisted of a Shimadzu HPLC, a HELEOS II MALS detector (Wyatt Technology) and a Superose® 6 Increase 10/300 GL column (cytiva). PBS was used as a running buffer with a flow rate of 0.5 mL/min and at a temperature of 20° C.10 to 100 μg of protein were injected. The Astra software v5 (Wyatt Technology) was used to collect and evaluate the data.

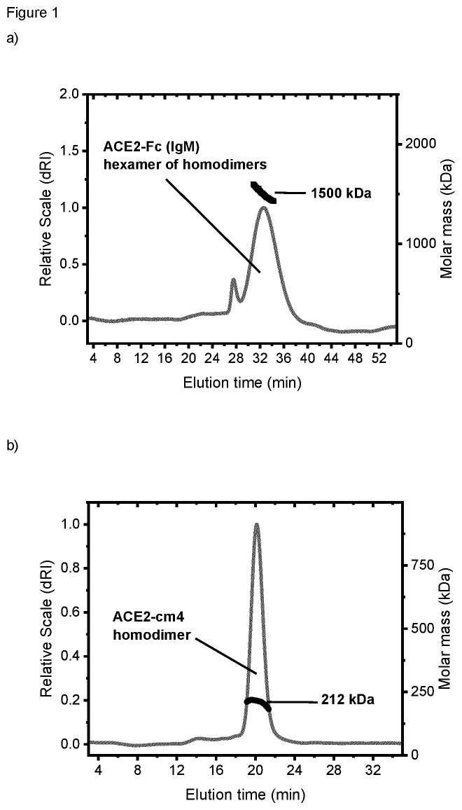

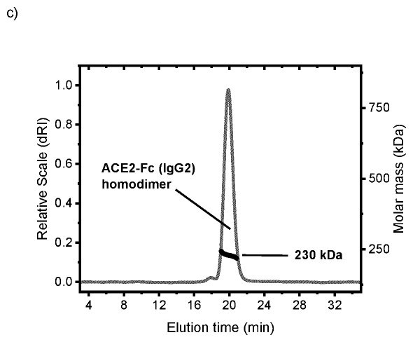

The ACE2-Fc (IgM) protein is more than 90% a hexamer of homodimers with a molecular mass of around 1,500 kDa (see FIGS. 1a and 3a). The ACE2-IgM-Fc homodimer peak gave a mass of about 250 kDa (FIG. 3a, right peak). Thus, the molecular mass of the ACE2-IgM-Fc hexamer fits to a complex composed of six homodimer units. The ACE2-Fc (cμ4) protein is more than 95% a homodimer with a molecular mass of around 212 kDa (see FIG. 1b). The ACE2-Fc (IgG2) protein is more than 95% a homodimer with a molecular mass of around 230 kDa (see FIG. 1c). Due to the large size of the ACE2-IgM-Fc hexamer, it was possible to calculate the average root mean square (rms) radius of the eluting protein which is 14.5±0.1 nm (FIG. 3b). This radius is very close to reported values for IgM hexamers (Keyt et al. (2020) Antibodies 9: 53).

### 2. Binding of the ACE2 Fusion Proteins to the Receptor-Binding Domain of SARS-CoV2

To determine if the ACE2-Fc fusion proteins can potently bind to the Spike protein receptor-binding domain (RBD) of SARS-CoV-2, Biacore experiments were performed. The ACE2-Fc binds with nanomolar affinity to the his-tagged Spike protein RBD of SARS-CoV-2 that is immobilized on CM5 chips.

A Biacore X-100 device was used for the measurements at room temperature. The ligand (SARS-CoV-2 RBD with a his-tag) was immobilized to approximately 100 RU on a CM5 chip. The running buffer was HBS-EP+. For the ACE2-cμ4 and ACE2-IgG2-Fc, the injected concentrations were 12.5, 25, 50, 100 and 200 nM. For the ACE2-IgM-Fc hexamer, the injected concentrations were 2.5, 5, 10, 20 and 40 nM. The sensorgrams were fit to a 1:1 binding model with the Biacore software to obtain the binding constants.

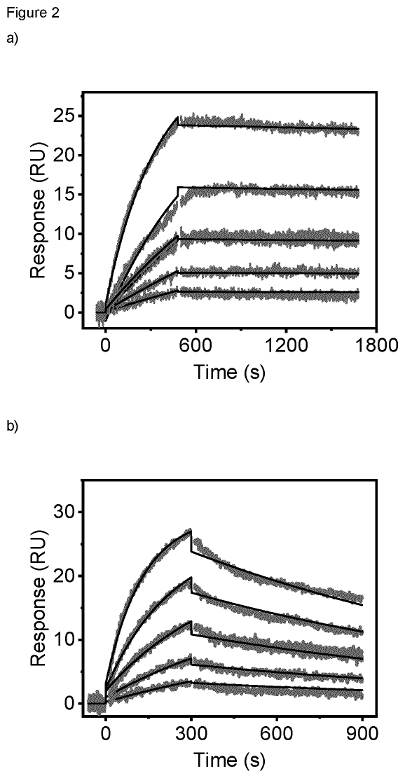

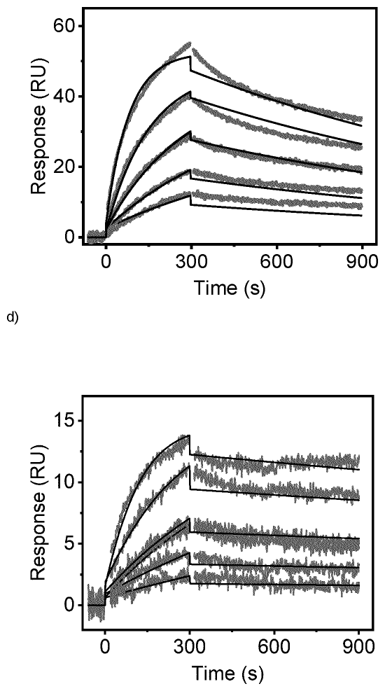

The ACE2-Fc (IgM) hexamer binds to the Spike protein receptor-binding domain (RBD) of SARS-CoV-2 with a binding constant of about 0.9 nM (see FIG. 2a). The ACE2-Fc (cμ4) protein binds to the Spike protein receptor-binding domain (RBD) of SARS-CoV-2 with a binding constant of about 20 nM (see FIG. 2b). The ACE2-Fc (IgG2) protein binds to the Spike protein receptor-binding domain (RBD) of SARS-CoV-2 with a binding constant of about 12.7 nM (see FIG. 2c). The ACE2-Fc (IgM) homodimer binds to the Spike protein receptor-binding domain (RBD) of SARS-CoV-2 with a binding constant of about 3.4 nM (see FIG. 2d).

3. Neutralization of the SARS-CoV2 Omicron Variant with the Hexameric ACE2-IgM Fusion Protein

The commercial pseudovirus neutralization assay was performed at Tebubio. HEK293 cells stably expressing the full-length ACE2 receptor (Genbank #NM_021804.3) and pseudotyped lentivirus produced with SARS-CoV-2 B.1.1.529 BA.1 variant Spike (Genbank #QHD43416.1 with B.1.1.529 BA.1 mutations) were used. These pseudovirions also contain the firefly luciferase gene driven by a CMV promoter, therefore, the spike-mediated cell entry can be conveniently measured via luciferase reporter activity. ACE2-HEK293 cells (BPS bioscience) were thawed, amplified in growth medium (BPS bioscience), then harvested and plated in white, clear flat bottom 96-well culture plates at 5×10³ cells/well in 50 μL of thaw medium. The cells were incubated overnight at 37° C. The following day, visual control of cell layer homogeneity and integrity was validated using inverted microscope. Test samples containing the ACE2-IgM fusion protein made of hexamers of homodimers of the polypeptide according to SEQ ID NO: 8 were diluted to reach 12× the expected final concentration in assay wells. The test samples were pre-incubated with Spike (SARS-CoV-2) pseudotyped lentivirus (BPS bioscience) or control bald pseudovirions (BPS bioscience) during approximately 30 minutes at room temperature. Finally, 10 μL of sample-pseudovirus mix were added in the assay wells containing the ACE2-HEK cells in 50 μL of medium. The IC50 was calculated in Prism v5 using a non-linear four parameter regression fit.

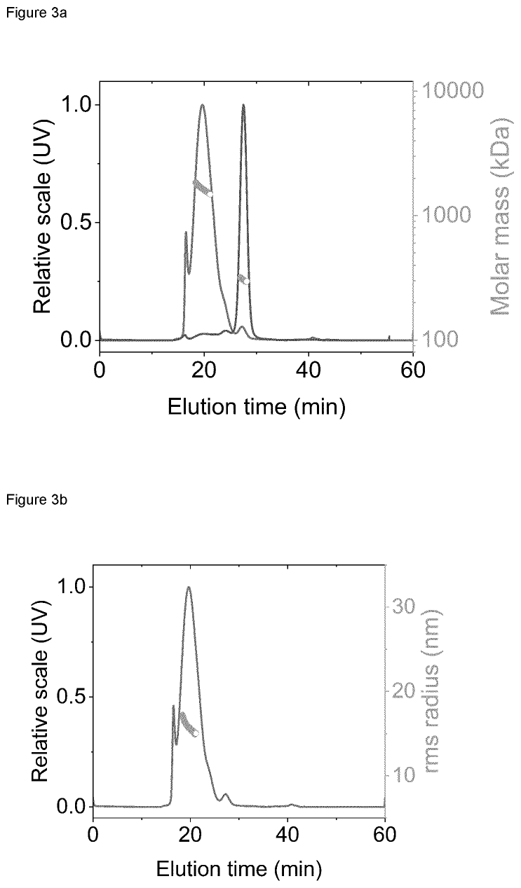

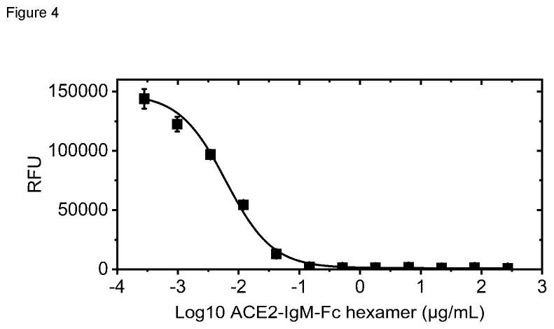

With this pseudovirus neutralization assay an IC50 of 6.6 ng/ml (4 pM) was observed (see FIG. 4) which further shows the virus neutralization potency of the hexameric construct.

### 4. In Vivo Testing of the ACE2-IgM Fusion Protein

Wild-type Syrian hamsters (Mesocricetus auratus) were purchased from Janvier Laboratories. Six- to eight-week-old female hamsters were used throughout the study.

Vero E6 (African green monkey kidney) cells were maintained in minimal essential medium (Gibco) supplemented with 10% fetal bovine serum (Integro), 1% bicarbonate (Gibco), and 1% L-glutamine (Gibco). All assays involving virus growth were performed using 2% fetal bovine serum.

The SARS-CoV-2 alpha (B.1.1.7) strain hCoV-19/Belgium/rega-12211513/2020 (EPI_ISL_791333, 2020-12-21) was isolated from nasopharyngeal swabs taken from travelers returning to Belgium in December 2020. The variant was subjected to sequencing on a MinION platform (Oxford Nanopore) directly from the nasopharyngeal swabs (Abdelnabi et al. (2021) EbioMedicine 68: 103403). The virus was cultured on Vero E6 cells and passage 2 virus was used for the study described here. The titer of the virus stock was determined by end-point dilution on Vero E6 cells by the Reed and Muench method (Reed et al. (1938) American Journal of Epidemiology 27: 493-497).

For in vivo antiviral efficacy, it was considered to be necessary to detect at least 1 log 10 reduction in viral RNA levels in treated subjects compared to the untreated, infected control group. Group size was calculated on the independent t-test with an effect size of 2.0 and a power of 80% (effect size=deltamean/SD=1 log 10 decrease in viral RNA/0.5 log 10), resulting in 5-6 animals/group.

The hamster infection model of SARS-CoV-2 was described before (Boudewijns et al., (2020) Nat. Comm. 11(1): 5838), Sanchez-Felipe et al. (2021) Nature 590(7845): 320-325). In brief, female Syrian hamsters (Mesocricetus auratus) 6-8 weeks of age were housed in individually ventilated cages (Sealsafe Plus, Tecniplast, cage type GR900) per two at 21° C., 55% humidity and 12:12-hour light:dark cycles. Hamsters were provided with food and water ad libitum as well as extra bedding material and wooden gnawing blocks. The animals were acclimated for four days prior to study start. A total of 38 golden Syrian hamsters were used in the study.

Prior to infection, the animals were anesthetized by intraperitoneal injection of a xylazine (16 mg/kg, XYL-M®, V.M.D.), ketamine (40 mg/kg, Nimatek, EuroVet), and atropine (0.2 mg/kg, Sterop) solution. Each animal was inoculated intranasally by gently adding 100 μL of virus stock containing 1×10⁴ TCID50 in both nostrils (50 μL per nostril). Animals were weighed prior to the start of the study and at study termination.

In the prophylactic setting, animals were treated with intranasal injections of 0.8 mg/kg or 1.7 mg/kg ACE2-IgM fusion proteins made of hexamers of homodimers of the polypeptide according to SEQ ID NO: 8 at equal volumes (50 μL per nostril) two hours prior to SARS-CoV-2 challenge and monitored until day four post infection (N=6 per group). In addition, hamsters were treated with 2.5 mg/kg Casirivimab (REGN10933 antibody) via the intranasal route at equal volumes (N=4). Control animals were treated with intranasal injections of PBS at equal volumes (50 μL per nostril) two hours prior to infection (N=6).

For histological examination, the lungs were fixed overnight in 4% formaldehyde and embedded in paraffin. Tissue sections (5 μm) were analyzed after staining with hematoxylin and eosin and scored blindly for lung damage by an expert pathologist. The scored parameters, to which a cumulative score of 1 to 3 was attributed (see Table 1), were the following: congestion, intra-alveolar hemorrhagic, apoptotic bodies in bronchus wall, necrotizing bronchiolitis, perivascular edema, bronchopneumonia, perivascular inflammation, peribronchial inflammation and vasculitis.

TABLE 1
Cumulative COVID-score

| Cumulative score | Severity of disease |
|---|---|
| 0 | None |
| 1 | Mild |
| 2 | Moderate |
| 3 | Severe |

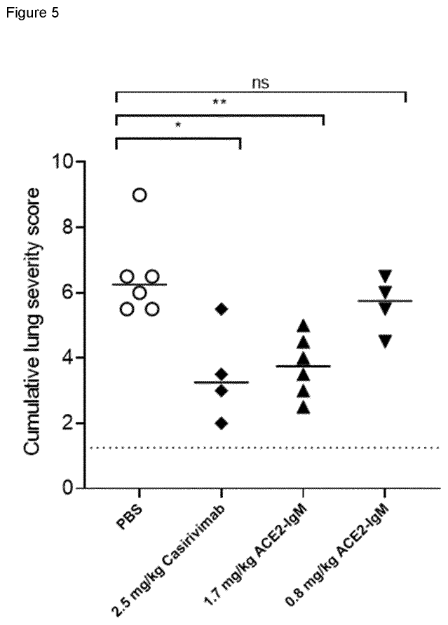

All six PBS control treated golden Syrian hamsters showed evidence of lung injury characterized by interstitial pneumonia with symptoms of perivascular and peribronchial inflammation and alveolar hemorrhage. All animals treated with Casirivimab via the intranasal route exhibited significantly improved lung pathology compared to the PBS-treated group at 4 days post infection (dpi) (see FIG. 5). Intranasal treatment of golden Syrian hamsters with ACE2-IgM made of hexamers of homodimers of the polypeptide according to SEQ ID NO: 8 in a prophylactic administration setting reduced the median cumulative lung score compared to PBS control-treated animals (see FIG. 5).

Some embodiments of the present invention relate to:

1. A fusion protein comprising a first part comprising a fragment of human ACE2 or a variant of said fragment, said human ACE2 having the amino acid sequence according to SEQ ID No. 1, and a second part comprising the Fc portion of human IgM or a fragment or variant of the Fc portion of human IgM.

2. Fusion protein according to item 1, wherein the Fc portion of human IgM has the amino acid sequence according to SEQ ID No. 4.

3. Fusion protein according to item 1 or 2, wherein the fragment of human ACE2 consists of the amino acid sequence according to SEQ ID No. 2.

4. Fusion protein according to item 1 or 2, wherein the fragment of human ACE2 is the extracellular domain of ACE2 consisting of the amino acid sequence according to SEQ ID No. 3.

5. Fusion protein according to any one of the preceding items, wherein the first part and the second part are linked by a linker sequence.

6. Fusion protein according to item 5, wherein the linker sequence consists of glycine and serine residues.

7. Fusion protein according to item 5 or 6, wherein the linker sequence has the amino acid sequence according to SEQ ID No. 5.

8. Fusion protein according to any one of the preceding items, further comprising a signal peptide.

9. Fusion protein according to item 8, wherein the signal peptide comprises the amino acid sequence according to SEQ ID No. 6.

10. Fusion protein according to any one of the preceding items, wherein the fragment of the Fc portion of human IgM consists of the amino acid sequence according to SEQ ID No 7.

11. Fusion protein according to any one of items 1, 2 and 4 to 9, having the amino acid sequence according to SEQ ID No. 8.

12. Fusion protein according to any one of items 1 and 4 to 10, having the amino acid sequence according to SEQ ID No. 9.

13. A fusion protein comprising a first part comprising a fragment of human ACE2 or a variant of said fragment, said human ACE2 having the amino acid sequence according to SEQ ID No. 1, and a second part comprising the Fc portion of human IgG2 or a variant of the Fc portion of human IgG2, wherein the first part and the second part are linked by a linker sequence.

14. Fusion protein according to item 13, wherein the Fc portion of human IgG2 comprises the amino acid sequence according to SEQ ID No. 10.

15. Fusion protein according to item 13 or 14, wherein the fragment of human ACE2 consists of the amino acid sequence according to SEQ ID No. 2.

16. Fusion protein according to item 13 or 14, wherein the fragment of human ACE2 is the extracellular domain of ACE2 consisting of the amino acid sequence according to SEQ ID No. 3.

17. Fusion protein according to any one of items 13 to 16, wherein the linker sequence consists of glycine and serine residues.

18. Fusion protein according to any one of items 13 to 17, wherein the linker sequence has the amino acid sequence according to SEQ ID No. 5.

19. Fusion protein according to any one of items 13, 14 and 16 to 18, having the amino acid sequence according to SEQ ID No. 11.

20. Fusion protein according to any one of items 1 to 10 and 13 to 18, wherein the variant of the human ACE2 fragment is an enzymatically inactive variant of human ACE2.

21. Fusion protein according to item 20, wherein the enzymatically inactive variant of human ACE2 comprises a H374N and a H378N mutation, the numbering referring to SEQ ID No. 1.

22. Fusion protein according to any one of items 1 to 10 and 13 to 18, wherein the variant of the human ACE2 fragment comprises an amino acid substitution at leucine 584, the numbering referring to SEQ ID No. 1.

23. Fusion protein according to any one of items 1 to 10 and 13 to 18, wherein the variant of the human ACE2 fragment comprises at least one amino acid substitution at at least one residue selected from lysine 619, arginine 621, lysine 625, arginine 697, lysine 702, arginine 705, arginine 708, arginine 710 and arginine 716, the numbering referring to SEQ ID No. 1.

24. Fusion protein according to any one of items 1 to 10 and 13 to 18, wherein the variant of the human ACE2 fragment comprises amino acid substitutions at lysine 619, arginine 621, lysine 625, arginine 697, lysine 702, arginine 705, arginine 708, arginine 710 and arginine 716, the numbering referring to SEQ ID No. 1.

25. Fusion protein according to any one of items 1 to 10 and 13 to 18, wherein the variant of the human ACE2 fragment comprises a S645C mutation, the numbering referring to SEQ ID No. 1.

26. Nucleic acid molecule comprising a nucleic acid sequence encoding the fusion protein according to any one of items 1 to 25.

27. Expression vector comprising the nucleic acid molecule according to item 26.

28. Host cell comprising the nucleic acid molecule according to item 26 or the expression vector according to item 27.

29. Method for producing the fusion protein according to any one of items 1 to 25, comprising culturing the host cell according to item 28 in a suitable culture medium.

30. A multimer formed by at least two molecules of the fusion protein according to any one of items 1 to 12.

31. Fusion protein according to any one of items 1 to 25 or multimer according to item 30 for medical use.

32. Fusion protein according to any one of items 1 to 25 or multimer according to item 30 for use in preventing and/or treating an infection with a coronavirus binding to ACE2.

33. Fusion protein or multimer for use according to item 32, wherein the coronavirus binding to ACE2 is selected from the group consisting of SARS, SARS-CoV-2 and NL63, preferably it is SARS-CoV-2.

34. Fusion protein or multimer for use according to item 32 or 33, wherein the fusion protein is to be administered in combination with an anti-viral agent.

35. Fusion protein or multimer for use according to item 34, wherein the anti-viral agent is selected from the group consisting of remdesivir, arbidol HCl, ritonavir, lopinavir, darunavir, ribavirin, chloroquin and derivatives thereof, nitazoxanide, camostat mesilate, tocilizumab, siltuximab, sarilumab and baricitinib phosphate.

36. Fusion protein according to any one of items 1 to 25 or multimer according to item 30 for use in treating hypertension (including high blood pressure), congestive heart failure, chronic heart failure, acute heart failure, contractile heart failure, myocardial infarction, arteriosclerosis, kidney failure, renal failure, Acute Respiratory Distress Syndrome (ARDS), Acute Lung Injury (ALI), chronic obstructive pulmonary disease (COPD), pulmonary hypertension, renal fibrosis, chronic renal failure, acute renal failure, acute kidney injury, inflammatory bowel disease and multi-organ dysfunction syndrome.

37. Fusion protein according to any one of items 1 to 25 or multimer according to item 30 for use in diagnosis.

38. Pharmaceutical composition comprising an effective amount of the fusion protein according to any one of items 1 to 25 or the multimer according to item 30 and a pharmaceutically acceptable carrier or excipient.

39. Pharmaceutical composition according to item 38, further comprising an anti-viral agent.

1. A fusion protein comprising a first part comprising a fragment of human ACE2 or a variant of said fragment, said human ACE2 having the amino acid sequence according to SEQ ID No. 1, and a second part comprising the Fc portion of human IgM or a fragment or variant of the Fc portion of human IgM.

2. The fusion protein of claim 1, wherein the Fc portion of human IgM has the amino acid sequence according to SEQ ID No. 4 or wherein the fragment of the Fc portion of human IgM consists of the amino acid sequence according to SEQ ID No 7.

3. The fusion protein of claim 1, wherein the fragment of human ACE2 consists of the amino acid sequence according to SEQ ID No. 2 or is the extracellular domain of ACE2 consisting of the amino acid sequence according to SEQ ID No. 3.

4. The fusion protein of claim 1, having the amino acid sequence according to SEQ ID No. 8 or 9.

5. A fusion protein comprising a first part comprising a fragment of human ACE2 or a variant of said fragment, said human ACE2 having the amino acid sequence according to SEQ ID No. 1, and a second part comprising the Fc portion of human IgG2 or a variant of the Fc portion of human IgG2, wherein the first part and the second part are linked by a linker sequence.

6. The fusion protein of claim 5, wherein the Fc portion of human IgG2 comprises the amino acid sequence according to SEQ ID No. 10.

7. The fusion protein of claim 5, wherein the fragment of human ACE2 consists of the amino acid sequence according to SEQ ID No. 2 or wherein the fragment of human ACE2 is the extracellular domain of ACE2 consisting of the amino acid sequence according to SEQ ID No. 3.

8. The fusion protein of claim 5, having the amino acid sequence according to SEQ ID No. 11.

9. The fusion protein of claim 1, wherein the variant of the human ACE2 fragment is an enzymatically inactive variant of human ACE2, preferably wherein the enzymatically inactive variant of human ACE2 comprises a H374N and a H378N mutation, the numbering referring to SEQ ID No. 1.

10. A nucleic acid molecule comprising a nucleic acid sequence encoding the fusion protein of claim 1.

11. An expression vector comprising the nucleic acid molecule of claim 10.

12. A host cell comprising the nucleic acid molecule of claim 10.

13. A multimer comprising at least two molecules of the fusion protein of claim 1.

14. A method of treating and/or preventing an infection with a coronavirus binding to ACE2 in a subject, the method comprising administering an effective amount of the fusion protein of claim 1 to the subject.

15. A pharmaceutical composition comprising an effective amount of the fusion protein of claim 1 and a pharmaceutically acceptable carrier or excipient.

16. The fusion protein of claim 5, wherein the variant of the human ACE2 fragment is an enzymatically inactive variant of human ACE2, preferably wherein the enzymatically inactive variant of human ACE2 comprises a H374N and a H378N mutation, the numbering referring to SEQ ID No. 1.

17. A method of treating and/or preventing an infection with a coronavirus binding to ACE2 in a subject, the method comprising administering an effective amount of the fusion protein of claim 5 to the subject.

18. A pharmaceutical composition comprising an effective amount of the fusion protein of claim 5 and a pharmaceutically acceptable carrier or excipient.

19. A method of treating and/or preventing an infection with a coronavirus binding to ACE2 in a subject, the method comprising administering an effective amount of the multimer of claim 13 to the subject.

20. A pharmaceutical composition comprising an effective amount of the multimer of claim 13 and a pharmaceutically acceptable carrier or excipient.
